SEQUENCE LISTING (2 sequences)

SEQ ID NO: 1 - protein, 708 aa, Homo sapiens
MTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGKLPGPAVEGPSATGPEIIRSPQDDASSPHLQVMLQIHLPGRHTLFVRAMIDSGASGNFIDHEYVAQNGIPLRIKDWPILVEAIDGRPIASGPVVHETHDLIVDLGDHREVLSFDVTQSPFFPVVLGVRWLSTHDPNITWSTRSIVFDSEYCRYHCRMYSPIPPSLPPPAPQPPLYYPVDGYRVYQPVRYYYVQNVYTPVDEHVYPDHRLVDPHIEMIPGAHSIPSGHVYSLSEPEMAALRDFVARNVKDGLITPTIAPNGAQVLQVKRGWKLQVSYDCRAPNNFTIQNQYPRLSIPNLEDQAHLATYTEFVPQIPGYQTYPTYAAYPTYPVGFAWYPVGRDGQGRSLYVPVMITWNPHWYRQPPVPQYPPPQPPPPPPPPPPPPSYSTL

SEQ ID NO: 2 - protein, 624 aa, Homo sapiens
MAENGESSGPPRPSRGPAAAQGSAAAPAEPKIIKVTVKTPKEKEEFAVPENSSVQQFKEAISKRFKSQTDQLVLIFAGKILKDQDTLIQHGIHDGLTVHLVIKSQNRPQGQSTQPSNAAGTNTTSASTPRSNSTPISTNSNPFGLGSLGGLAGLSSLGLSSTNFSELQSQMQQQLMASPEMMIQIMENPFVQSMLSNPDLMRQLIMANPQMQQLIQRNPEISHLLNNPDIMRQTLEIARNPAMMQEMMRNQDLALSNLESIPGGYNALRRMYTDIQEPMLNAAQEQFGGNPFASVGSSSSSGEGTQPSRTENRDPLPNPWAPPPATQSSATTSTTTSTGSGSGNSSSNATGNTVAAANYVASIFSTPGMQSLLQQITENPQLIQNMLSAPYMRSMMQSLSQNPDLAAQMMLNSPLFTANPQLQEQMRPQLPAFLQQMQNPDTLSAMSNPRAMQALMQIQQGLQTLATEAPGLIPSFTPGVGVGVLGTAIGPVGPVTPIGPIGPIVPFTPIGPIGPIGPTGPAAPPGSTGSGGPTGPTVSSAAPSETTSPTSESGPNQQFIQQMVQALAGANAPQLPNPEVRFQQQLEQLNAMGFLNREANLQALIATGGDINAAIERLLGSQPS

(19) United States
(12) Patent Application Publication
(10) Pub. No.: US 2023/0167501 A1
(43) Pub. Date: Jun. 1, 2023

(54) Methods For The Identification Of UBQLN2-Mediated Amyotrophic Lateral Sclerosis (ALS)

(71) Applicant: The Regents of the University of Colorado, a body corporate, Denver, CO (US)

(72) Inventors: Alexandra Whiteley, Boulder, CO (US); Shannon N. Leslie, Lafayette, CO (US)

(21) Appl. No.: 17/822,992

(22) Filed: Aug. 29, 2022

Related U.S. Application Data
(60) Provisional application No. 63/238,652, filed on Aug. 30, 2021.

(51) Int. Cl. C12Q 1/6883; G01N 33/68

(52) U.S. Cl. CPC C12Q 1/6883; G01N 33/6893; C12Q 1/6869

(57) ABSTRACT

The current invention includes novel methods and compositions for the detection of novel biomarkers of ALS.

### CROSS-REFERENCE TO RELATED APPLICATIONS

This U.S. nonprovisional application claims priority to, and the benefit of U.S. Provisional Application No. 63/283,652, filed Aug. 30, 2021, which is incorporated herein by reference in its entirety.

### SEQUENCE LISTING

The instant application contains contents of the electronic sequence listing (90245-00671-Sequence-Listing.xml; Size: 4,130 bytes; and Date of Creation: Aug. 29, 2022) is herein incorporated by reference in its entirety.

### TECHNICAL FIELD

The present invention relates to methods and materials involved in assessing biological samples for the presence or levels of a biomarker. For example, this document provides methods and materials for determining whether or not a biological sample contains a level of a certain biomarker for amyotrophic lateral sclerosis (ALS) in a subject.

### BACKGROUND

ALS is a fatal neuromuscular disorder characterized by progressive paralysis that affects upwards of 30,000 Americans. It is a devastating disease that afflicts people, primarily in the fifth to sixth decade of life, and typically kills within 3-5 years of diagnosis. There is no cure for ALS, and there are no reliable biomarkers for the disease and few treatment options—none of which significantly prolong lifespan. Two leading therapeutics for ALS are Riluzole and Edaravone, which have shown limited therapeutic success. Both work by inhibiting excitotoxic glutamatergic signaling and oxidative stress, respectively. However, these drugs have shown limited benefit in reducing symptoms and do not significantly lengthen lifespan. There is a significant unmet medical need for novel ALS therapeutics.

Historically, neurodegenerative diseases involving protein buildup have been challenging to target. There are multiple, high-profile examples of biologic therapies (such as the monoclonal antibodies gantenerumab and solanezumab) intended to facilitate degradation of protein aggregates in Alzheimer's which have been successful to varying degrees at clearing the aggregates, but having not demonstrated major improvement in symptoms or disease progression. Other ALS drugs that work downstream of protein aggregation have significant side effects due to the fact that they alter generalized neuronal function. For example, Riluzole inhibits excitotoxicity, which is thought to contribute to the symptoms of ALS due to neuronal dysfunction. However, Riluzole is often poorly tolerated, resulting in elevated liver enzymes and rarely can cause serious interstitial lung disease, especially in people of Japanese descent.

One of the major roadblocks to developing ALS therapies is that very little is known about the molecular mechanism of disease. In general, ALS shares a few key molecular perturbations. ALS patient tissues exhibit protein accumulation and aggregation, which is thought to contribute to neuronal dysfunction. However, the specifics of how aggregates lead to neuronal dysfunction are poorly understood. There are multiple genes that have been identified as risk factors for a familial form of ALS (fALS), which accounts for approximately 10% of total ALS cases; the other cases are considered sporadic (sALS), with no known genetic etiology. While there are multiple unique genetic mutations that can lead to fALS, each genetic mutation is unique, current treatment options therapeutically target downstream effects that are shared amongst familial and sporadic ALS.

UBQLN2 is a known genetic risk factor for ALS. Mutations in UBQLN2 cause approximately 2% of familial ALS cases, but the mechanism of disease was not known and may be shared broadly amongst other forms of ALS. The present inventors demonstrate a novel molecular pathway from UBQLN2 mutation to the development of disease through the discovery that UBQLN2 regulates the abundance of a virus-like protein called PEG10. PEG10 resembles a retroviral protease, and when it accumulates in the cell upon UBQLN2 dysfunction, proteolytic activity results in a transcriptional stress response that resembles the neuronal dysfunction present in ALS (FIGS. 1-6). Therefore, PEG10 is a promising diagnostic biomarker for the diagnosis and/or early intervention of ALS.

### SUMMARY OF THE INVENTION

One aspect of the current invention includes novel methods and compositions for the detection of PEG10 in a biomarker of ALS. As described herein, PEG10 has been demonstrated to accumulate upon UBQLN2-mediated neurodegenerative disease and sporadic ALS (FIGS. 6, 10-12) and that its accumulation increases enzymatic activity. Indeed, the present inventors' discovery that the PEG10 enzyme is active upon accumulation in the cell and that the accumulation only occurs when UBQLN2 is dysfunctional is entirely novel in the field. This PEG10 enzymatic activity directly contributes to the development of neurodegenerative disease initiation and/or progression.

In one preferred aspect, the invention includes novel methods and compositions for the detection and early treatment of neurodegenerative disease, and preferably ALS, by detecting the levels and/or activity of PEG10 in a subject in need thereof.

One aspect of this disclosure provides methods related to predicting a subjects risk and/or response to an ALS treatment regimen comprising an anti-ALS agent, using the expression levels of the functional biomarker identified by the inventors as correlated with a ALS patient's likelihood of response (or lack of response) to the ALS treatment regimen. These expression levels may be assessed at the protein levels (protein content, protein activity, gain of function or loss of function mutations, prolonged protein half-life, post translational modifications, etc.).

One embodiment of this aspect of the disclosure is a method that includes determining the expression level of a biomarker in a cell from a ALS patient, being defines herein as a subject, and preferably a human subject, at risk of developing ALS, or having symptoms of ALS, or having a formal diagnosis of ALS. The biomarker PEG10 may be detected and may follow treatment with an ALS treatment such as riluzole or edaravone.

Another embodiment of this aspect of the disclosure is a method that includes determining, the expression level of the PEG10 biomarker. The level of PEG10 that is greater than a reference number may then be correlated with an increased likelihood that the subject has, or is at risk of developing ALS, and if the ALS subject will respond to an early treatment regimen including an anti-ALS agent such as riluzole or edaravone.

Another embodiment of this aspect of the disclosure is a method that includes assaying a biological sample from a subject for the level of a biomarker, and preferably the level of PEG10 accumulated in the sample. In these embodiments, the biomarker is detected by a method selected from Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, proteomic mass spectrometry, microcytometry, microarray, microscopy, fluorescence activated cell sorting (FACS), and flow cytometry. In some embodiments, the biomarker is detected by immunohistochemical (IHC) analysis.

In these assay kits, the means for detecting the biomarkers may include a nucleotide probe that hybridizes to a portion of the biomarker mRNA. Alternatively or additionally, in these assay kits, the means for detecting the biomarkers may include a detectable label. In certain embodiments, in these assay kits the means for detecting is immobilized on a substrate.

This Summary of the Invention is neither intended nor should it be construed as being representative of the full extent and scope of the present invention. Moreover, references made herein to “the present invention,” or aspects thereof, should be understood to mean certain embodiments of the present invention and should not necessarily be construed as limiting all embodiments to a particular description. The present invention is set forth in various levels of detail in the Summary of the Invention as well as in the attached drawings and the Description of Embodiments and no limitation as to the scope of the present invention is intended by either the inclusion or non-inclusion of elements, components, etc. in this Summary of the Invention. Additional aspects of the present invention will become more readily apparent from the Description of Embodiments, particularly when taken together with the drawings.

### BRIEF DESCRIPTION OF THE FIGURES

FIGS. 1A-H: UBQLN2 regulates PEG10 gag-pol abundance. (A) Human ESCs had individual UBQLN genes deleted by CRISPR gene editing and clones were probed by western blot for endogenous PEG10 protein. Full-length gag-pol protein accumulates only upon UBQLN2 loss. n=3 independent experiments. (B-C) Quantification of gag-pol (B) and gag (C) abundance in hESC cell lines of (A). PEG10 protein was normalized to Tubulin, then normalized to the average intensity for each individual experiment. n=3 independent experiments, and significance was determined by multiple comparisons test. No differences in gag (C) were detected with an ordinary one-way ANOVA. (D) Schematic of PEG10 protein. The first reading frame (gag) contains a capsid-like (CA) region, as well as a retroviral zinc finger (‘CCHC’). The pol-like sequence contains a retroviral-type aspartic protease with one active site ‘DSG’ motif, as well as a C-terminal polyproline repeat domain (PPR). (E) Schematic of PEG10 protein abundance reporter. PEG10 is fused at the 3′ end to Dendra2, followed by an IRES-CFP. Right: example dot plot showing Dendra2 and CFP signal in transfected cells. (F) Dendra2 over CFP MFI ratio for PEG10 in WT and UBQLN1, 2, and 4 ‘TKO’ HEK293 cells. Shown is mean±SEM of MFI ratio from four independent experiments with triplicate transfection wells. Unpaired comparison between WT and TKO Dendra2/CFP ratios was performed by Student's t-test. (G) WT and truncation mutant of PEG10 gag-pol fused to the fluorophore Dendra2. ΔPPR is missing the last 27 amino acids containing polyproline repeat. (H) Protein abundance of PEG10-Dendra2 fusions was determined for WT and TKO cells by flow cytometry. Values over 1.0 indicate dependence on UBQLNs for restriction. n=5 independent experiments.

FIGS. 2A-E: PEG10 self-cleaves to generate fragments with unique localization. (A) Mutation of the active site aspartic acid in the protease domain results in disappearance of cleaved PEG10 products. N-terminally HA-tagged PEG10 was expressed either as WT or ‘ASG’ protease mutant in cells and probed by western blot for HA. Based on estimated molecular weight, the fragments are estimated to encompass gag-capsid (CA) and gag-capsid (NTD) (CA^{NTD}) fragments. n=4 independent experiments. (B) Model of PEG10 self-cleavage. PEG10 cleaves gag to generate a liberated nucleocapsid (NC) fragment. PEG10 also cleaves the gag-CA domain into CA^{NTD} and CA^{CTD}. Dotted lines indicate that the fragments are not visible by western blot due to the absence of the N-terminal HA tag. (C) Presence of cleaved PEG10 products in virus-like particles (VLPs). VLPs were isolated from PEG10-transfected HEK cells by ultracentrifugation and probed for cleavage products by western blot. Tubulin was used as a control for contamination of conditioned medium with cell fragments. n=3 independent experiments. (D) PEG10 gag is capable of being cleaved by PEG10 gag-pol in trans. HA-tagged and PEG10-Dendra2 fusion constructs were co-transfected into cells and the presence of HA-tagged cleavage products was assessed by western blot. n=2 independent experiments. (E) Localization of PEG10 fragments. Cells were transfected with HA-tagged PEG10 constructs and imaged by confocal microscopy after staining with an HA antibody and DAPI. Scale bar 10 Shown are representative cells from 10 fields of view of each construct. n=3 independent experiments.

FIGS. 3A-F: Liberated nucleocapsid alters transcription of axon extension genes. (A) Cluster profiling of gene expression effects as measured by RNA-Seq analysis upon PEG10 construct overexpression. The number of genes in each group is listed in parentheses. Data are shown as box and whiskers min to max with line at median. (B) Normalized counts of TXNIP transcript from RNA-Seq analysis of three biological replicates from PEG10 transfected or control transfected cells. (C) Top gene expression changes in NC-transfected cells by GO-term enrichment analysis. The top five GO-terms ranked by adjusted p value are shown. Adjusted p value is shown by color, and size of datapoint reflects the number of genes enriched in the pathway. (D) Heatmap of genes from the Axon extension GO-term showing Row z-score for each gene in the pathway. (E) Normalized counts of DCLK1 from RNA-Seq analysis of PEG10-transfected cells. (F) RNA-Seq data from the Target ALS dataset of post-mortem lumbar spinal cords were analyzed for DCLK1 (left) and TXNIP (right) counts. NNC=non-neurological control. For (B,E), data are shown as min-max floating bars with line at mean. For (F), data are shown as 5-95% box and whisker plot and significance was determined by DESeq2.

FIGS. 4A-C: ALS-causing UBQLN2 mutation can influence PEG10 gag-pol levels. (A) WT or TKO cells stably transfected with doxycycline- (dox-) inducible constructs expressing Myc-UBQLN2, Myc-UBQLN2^{P497H}, or Myc-UBQLN2^{P506T} were probed for endogenous PEG10. (B) Quantitation of gag-pol abundance in mutant UBQLN2-expressing cells. Gag-pol was normalized to Tubulin and to the average intensity of each experiment. Mean±SEM is shown for each condition. n=5 wells per condition collected from two different passages. (C) Quantitation of gag abundance. Shown is mean±SEM of n=5 wells. Multiple comparison tests were run with Bonferroni correction to compare WT and TKO cells as well as WT OE with the two mutant lines.

FIGS. 5A-D: PEG10 gag-pol protein accumulates in human ALS. (A) Schematic illustrating multiplexed global proteomic strategy to quantify PEG10 protein from human lumbar spinal cord. Two non-neurological controls (NNC), one fALS case with a UBQLN2 mutation, and six sporadic ALS cases were combined with a ‘spike-in’ PEG10 channel containing 5% lysate from cells transfected with HA-PEG10 gag-pol and 95% spinal cord lysate to normalize proteomic background complexity. All ten samples were labeled with tandem mass tags (TMT) and run as a 10-plex on LC-MS2. (B-C) Abundance of PEG10 gag (B), and pol (C), in human spinal cord. Significance was determined by Student's t-test. (D) Global proteomic analysis with 7,465 individual proteins quantified. All ALS samples were grouped together, and two non-neurological controls (NNC) were grouped to generated log₂ ratio of protein abundance and significance calculation by homoscedastic unpaired t-test. PEG10 pol is highlighted in blue, and PEG10 gag (not significant) is highlighted in pink.

FIG. 6: Antibodies used in this present application.

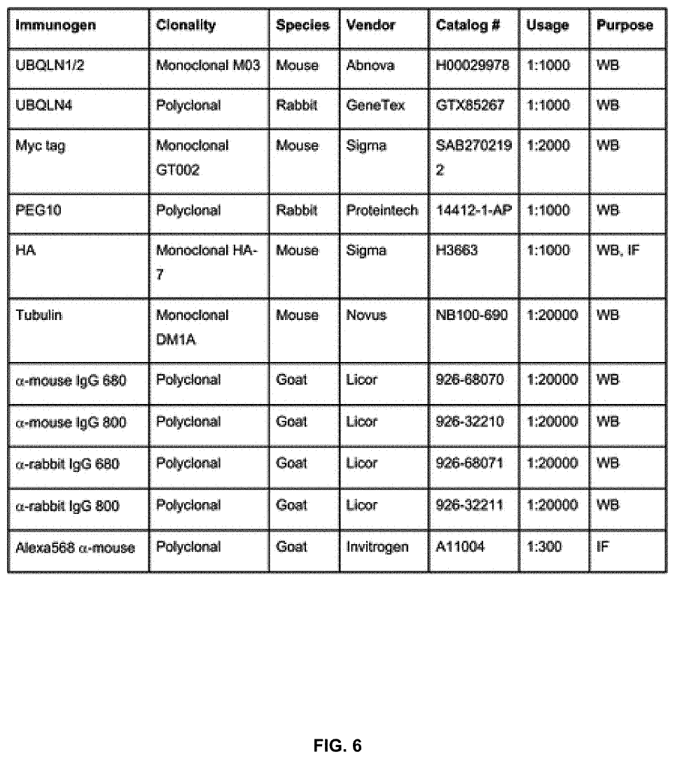

FIG. 7: Global proteomics data from mouse spinal cord demonstrating that PEG10 protein levels are elevated in spinal cord of Ubqln2−/− mice. Lumbar spinal cord (n=6 Ubqln2−/− and 4 WT) was isolated from 4-month-old animals for TMT analysis. Information on the total number of proteins identified and quantified in each TMT.

FIGS. 8A-E: Abundance of PEG10 is changed in ALS at the protein, but not the mRNA, level. (A-B) Schematic of PEG10 protein from gag (A) and pol (B) with regions highlighted where peptides were quantified by global proteomic analysis. Black: Non-neurological control, blue: UBQLN2-fALS, purple: sporadic ALS. Peptide abundance as measured by mass spectrometer is shown on y axis, with mean±SEM of replicates. (C) RNA-Seq counts of PEG10 from post-mortem lumbar spinal cord tissue of patients with Classical ALS or Non-Neurological Controls (NNC). (D) Quantification of neurogranin (y axis) and PEG10 pol peptides (x axis) are plotted for all 9 spinal cord samples to demonstrate the relationship between the two markers. (E) Quantification of neurofilament medium (y axis) and PEG10 pol peptides (x axis) are plotted for all 9 spinal cord samples to demonstrate the relationship between the two markers.

FIG. 9: Frozen section of post-mortem lumbar spinal cord was stained for PEG10, MAP2, and DAPI. PEG10 (green) is in the ventral horns of spinal cord. MAP2 is red, DAPI is blue. Scale bar 1 mm. Right: Quantitation of PEG10 intensity in sections in Arbitrary Units as defined by FIJI software.

### DETAILED DESCRIPTION OF THE INVENTION

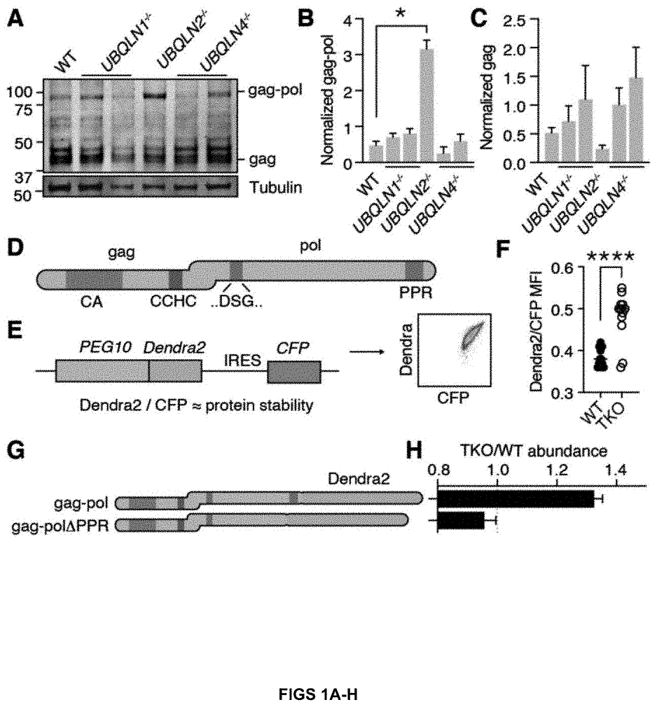

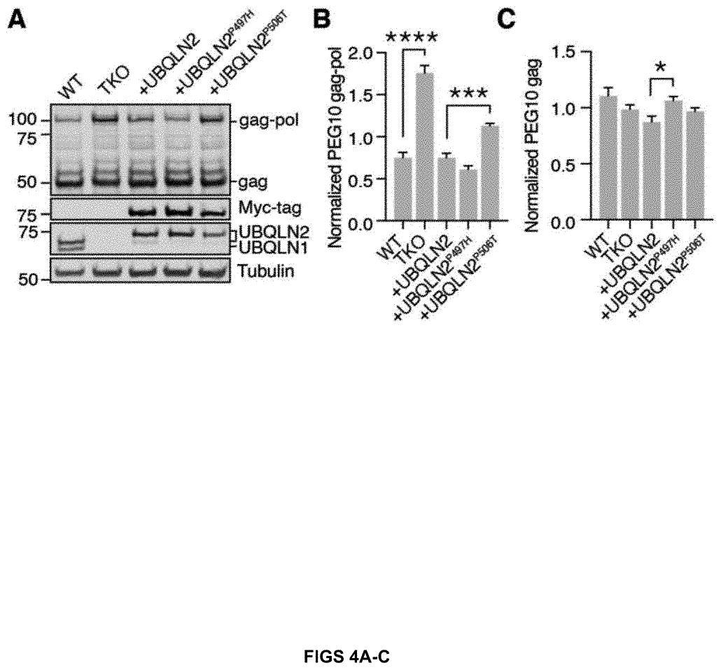

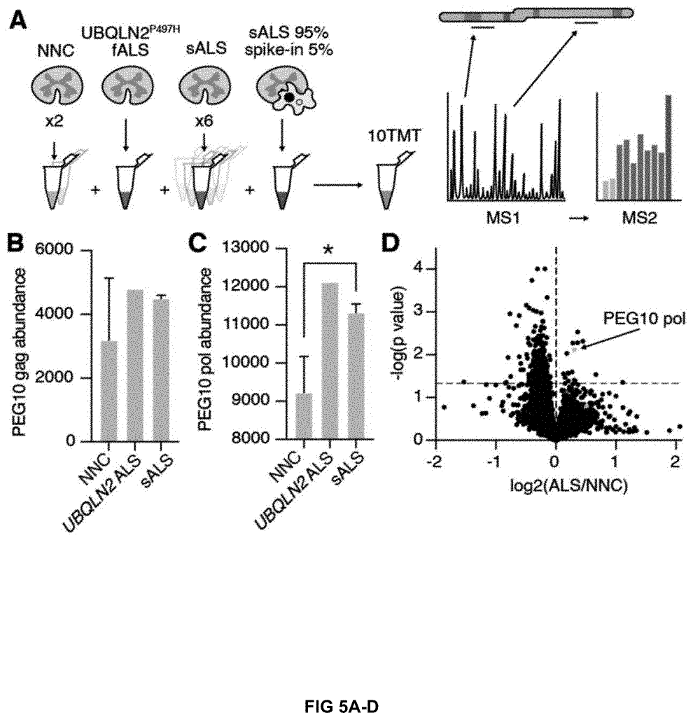

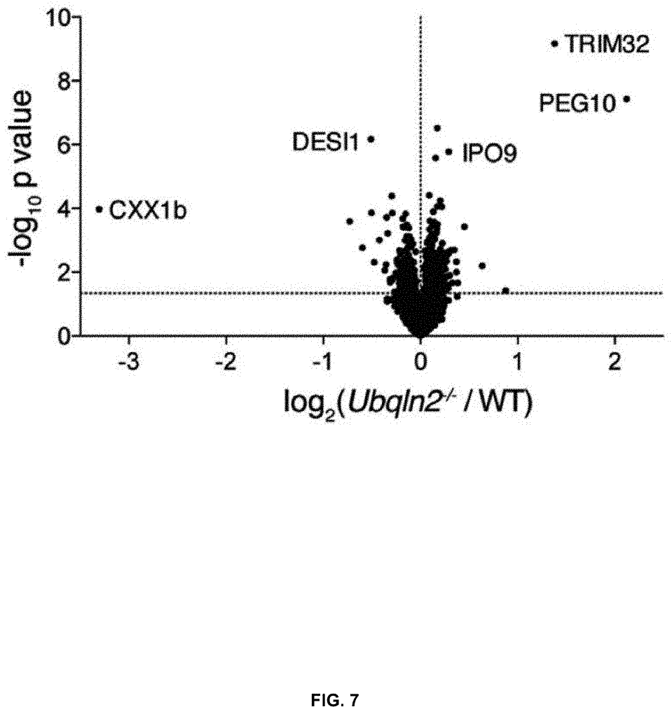

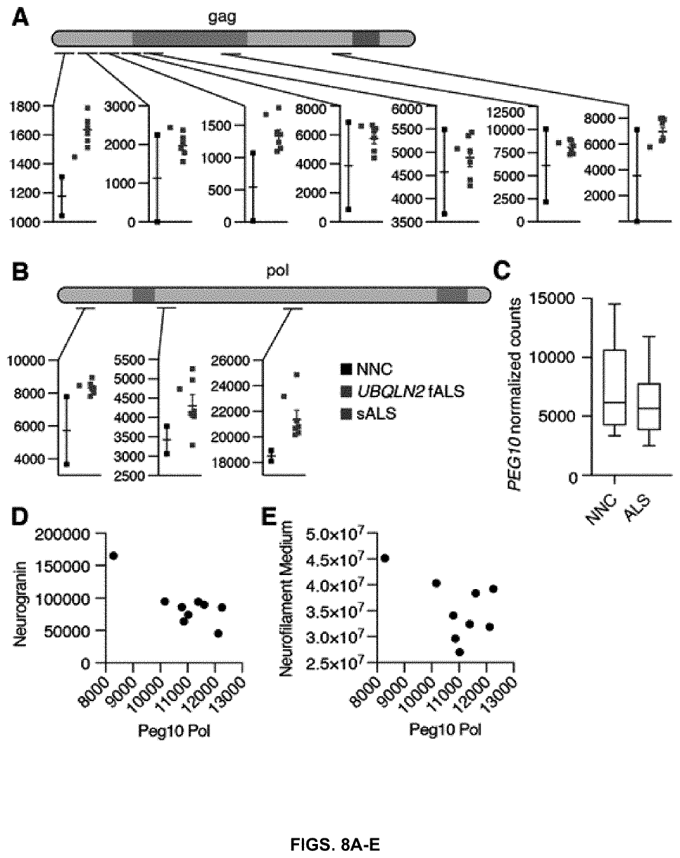

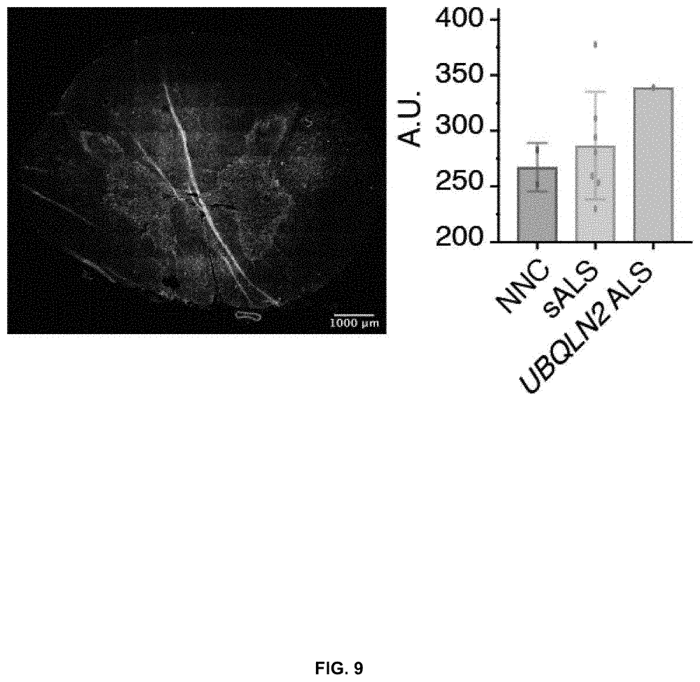

As noted above, ubiquilins (UBQLNs), a family of proteins that facilitate protein degradation by the proteasome, play a major role in the disease progression of ALS. Mutations in UBQLN2 (SEQ ID NO. 2) cause ALS in humans, but how UBQLN2 dysfunction leads to this devastating and fatal neurodegenerative disease remains poorly understood. Prior studies have shown that only a limited number of proteins dramatically accumulated in UBQLN2-deficient tissues. As shown in FIGS. 1 and 7, the present inventors have shown that one protein shown to accumulate in UBQLN2-deficient or UBQLN2-mutant cells (FIG. 1A-C, FIG. 4) and tissues (FIGS. 5, 7-9) is PEG10, and may directly contribute to disease.

In one embodiment of the invention, the present inventors demonstrate that the quantity and/or the activity of PEG10 (SEQ ID NO. 1) is diagnostically relevant to ALS in a subject in need thereof. PEG10 is a poorly understood gene that resembles HIV but is not infectious. As shown generally in above, PEG10 has a retrovirus-like gag region with a functional RNA binding domain, and a pol region with a retroviral aspartic protease, followed by a long C-terminus that renders it dependent on UBQLN2 for degradation. The gag and pol regions are separated by a stop codon, but due to virus-like ribosome frameshifting, a short gag and long gag-pol form of PEG10 are both made. UBQLN2 facilitates degradation of only the gag-pol, protease-containing form.

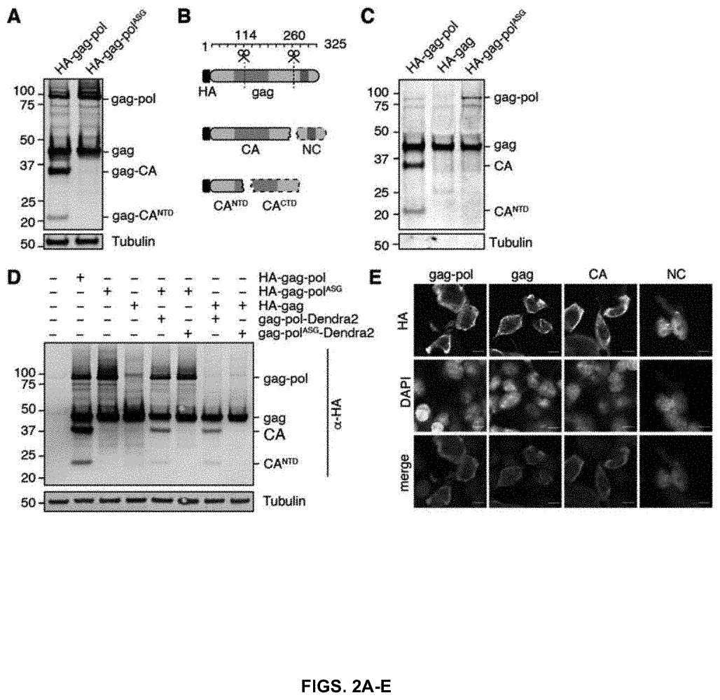

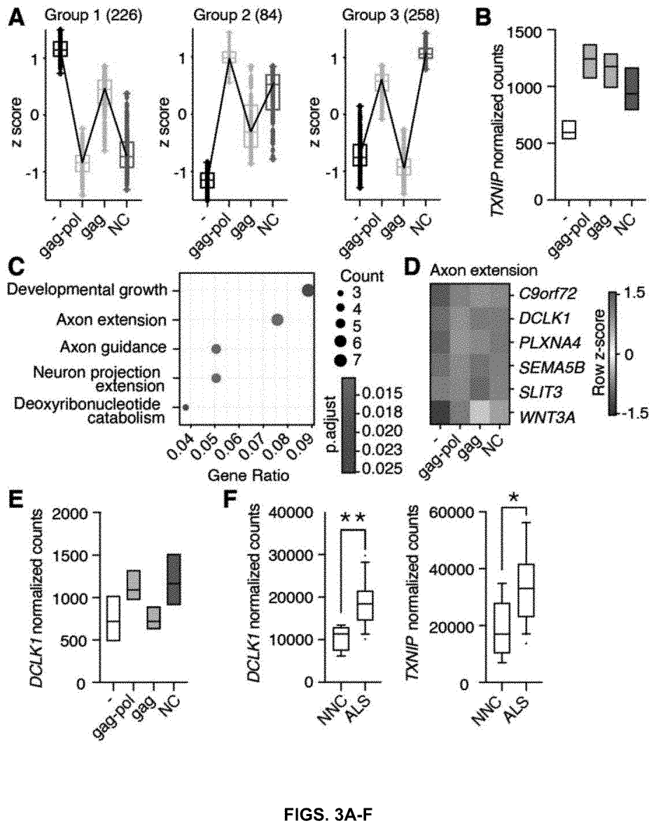

As demonstrated in FIGS. 2-3, the present inventors have shown that PEG10 gag-pol cleaves itself to generate a small RNA-binding fragment which then moves to the nucleus and changes gene expression in the cell. Such action demonstrates a model for UBQLN2 dysfunction that may lead to neurodegenerative disease. Like TDP43 and FUS mutations, which account for a considerable fraction of familial ALS cases, the self-cleavage of PEG10 changes gene expression which may contribute to neuron dysfunction and loss.

As described herein, PEG10 has been demonstrated to accumulate upon UBQLN2-mediated neurodegenerative disease and that its accumulation increases enzymatic activity. Indeed, the present inventors' discovery that the PEG10 enzyme's activity increases upon its accumulation in the cell and that the accumulation only occurs when UBQLN2 is dysfunctional is entirely novel in the field. This PEG10 enzymatic activity directly contributes to the development of neurodegenerative disease initiation and/or progression.

The inventive technology may include one or more markers that may be used for diagnostic purposes, as well as for drug screening and therapeutic purposes as well as other purposes described herein. Markers may include, but not be limited to the PEG10 and/or UBQLN2 protein, or fragments thereof.

Accordingly, one embodiment of the present invention relates to a method and corresponding assay kit for use to select a ALS patient, or a patient that is at risk of developing ALS, who can benefit from therapeutic administration of an anti-ALS compound. The method generally includes detecting in a biological sample, such as cerebral-spinal fluid or spinal or neuronal cells, from a patient the biomarker PEG10 and/or UBQLN2 protein, or fragments thereof, such as the gag or pol portions.

The present inventors have discovered that patients with ALS cells displaying high levels of accumulated PEG10 protein have, or are susceptible to developing ALS and can benefit from anti-ALS compounds. Levels of protein expression of PEG10 and/or UBQLN2 may be assessed by immunohistochemistry. In one embodiment, high levels of PEG10 protein are statistically significantly associated with better response, disease control rate, time to progression and survival following treatment with these ant-ALS compounds.

The methods and test kits provided by the present invention are extremely useful for patients susceptible to ALS that can be treated with at least one anti-ALS compounds. Such patients might, as a result of the methods provided herein, be able to receive early anti-ALS interventions.

In one or more embodiments of the invention, the methods include the detection of PEG10 and/or UBQLN2 proteins using immunohistochemistry (IHC) or proteomics techniques. Notably, the invention is not limited to the detection techniques described herein (e.g., IHC), since other techniques may be used to achieve the same result.

The methods of the present invention include detecting in a biological sample from a patient to be tested for the levels of PEG10 and/or UBQLN2 protein in the sample.

Suitable methods of obtaining a biological sample from a patient are known to a person of skill in the art. A patient sample can include any bodily fluid or tissue from a patient that may contain PEG10 protein, and may preferably include neuronal cells, tissues or fluids, such as CSF fluid. More specifically, according to the present invention, the term “biological sample,” or “test sample,” “subject sample” or “patient sample” can be used generally to refer to a sample of any type which contains cells or products that have been secreted from cells to be evaluated by the present method, including but not limited to, a sample of isolated cells, a tissue sample and/or a bodily fluid sample. Most typically in the present invention, the sample is a bodily fluid sample, such as CSF bodily fluid, or a cell or tissue from a spinal or brain sample. According to the present invention, a biological sample of ALS cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. The cells in the cell sample are not necessarily of the same type, although purification methods can be used to enrich for the type of cells that are preferably evaluated. Cells can be obtained, for example, by scraping of a tissue, processing of a tissue sample to release individual cells, or isolation from a bodily fluid.

A tissue sample, although similar to a sample of isolated cells, is defined herein as a section of an organ or tissue of the body which typically includes several cell types and/or cytoskeletal structure which holds the cells together. One of skill in the art will appreciate that the term “tissue sample” may be used, in some instances, interchangeably with a “cell sample”, although it is preferably used to designate a more complex structure than a cell sample. A tissue sample can be obtained by a biopsy, for example, including by cutting, slicing, or a punch.

A bodily fluid sample, like the tissue sample, contains the cells to be evaluated, and is a fluid obtained by any method suitable for the particular bodily fluid to be sampled. Bodily fluids suitable for sampling may preferably include blood, or CSF fluid.

The protein expression in a biological sample of ALS cells according to the invention can be measured, for example in immunohistochemistry assays, in cell nuclei, cytoplasm and/or membranes. Immunohistochemistry, as well as other detection methods, can be performed in blood or spinal fluid, among others. The markers can be measured in specimens that are fresh, frozen, fixed or otherwise preserved.

Once a sample is obtained from the patient, the sample is evaluated for detection of the biomarker described herein. In some embodiments of the present invention, a tissue, a cell or a portion thereof (e.g., a section of tissue, a component of a cell such as nucleic acids, etc.) is contacted with one or more nucleic acids. Such protocols are used to detect gene expression, gene amplification, and/or gene polysomy, for example. Such methods can include cell-based assays or non-cell-based assays. The tissue or cell expressing a target gene is typically contacted with a detection agent (e.g., a probe, primer, or other detectable marker), by any suitable method, such as by mixing, hybridizing, or combining in a manner that allows detection of the target gene by a suitable technique.

The patient sample is prepared by any suitable method for the detection technique utilized. In one embodiment, the patient sample can be used fresh, frozen, fixed or otherwise preserved. For example, the patient cells can be prepared by immobilizing patient tissue in paraffin. The immobilized tissue can be sectioned and then contacted with a probe for detection of hybridization of the probe to a target gene.

In the method of the invention, the level of PEG10 or UBQLN2 protein expression in the test sample is compared to a control level of that PEG10 or UBQLN2 expression selected from: (i) a control level that has been correlated with sensitivity to an anti-ALS compound of the invention; and (ii) a control level that has been correlated with resistance to an anti-ALS compound of the invention; (iii) a control level that has been correlated with non-development of ALS. A patient is selected as being predicted to benefit from therapeutic administration of the anti-ALS compound of the invention, an agonist thereof, or a drug having substantially similar biological activity, if the level of protein expression in the patient's ALS cells is statistically higher to the control level of protein expression that has been correlated with lack of ALS development. A patient is predicted to not benefit from therapeutic administration of an anti-ALS compound of the invention, an agonist thereof, or a drug having substantially similar biological activity as an anti-ALS compound of the invention, if the levels of PEG10 protein in the patient's sample is statistically the same or less than the control level of protein expression that has been correlated with a non-development ALS in a subject.

By “control sample” is meant a cell, cell sample, or protein or DNA sample that is used to test the levels or activity of PEG10 and/or UBQLN2. For example, a “control sample” can be a biological sample, such as a cell sample, or protein or DNA sample that is used as a reference. For example, in experiments to determine the levels or activity of PEG10 and/or UBQLN2, the control sample may be a non-ALS biological specimen (e.g., a non-ALS cell from a patient) or a lysate prepared from such a cell. A “control level” or “control” may be determined by testing or assaying a control sample. According to the present invention, a control level is a level of protein expression, which can include a level that is correlated with “wild-type” levels of expression or activity PEG10 or UBQLN2 which do not result in the development of ALS, or ALS-like symptoms.

The method for establishing a control level of expression is selected based on the sample type, the tissue or organ from which the sample is obtained, and the status of the patient to be evaluated. Preferably, the method is the same method that will be used to evaluate the sample in the patient. In a preferred embodiment, the control level is established using the same cell type as the cell to be evaluated. In a preferred embodiment, the control level is established from control samples that are from patients or cell lines known to not be susceptible to, or have ALS. In one aspect, the control samples are obtained from a population of matched individuals. According to the present invention, the phrase “matched individuals” refers to a matching of the control individuals on the basis of one or more characteristics which are suitable for the type of cell or characteristic to be evaluated. For example, control individuals can be matched with the patient to be evaluated on the basis of gender, age, race, or any relevant biological or sociological factor that may affect the baseline of the control individuals and the patient (e.g., preexisting conditions, consumption of particular substances, levels of other biological or physiological factors). To establish a control level, samples from a number of matched individuals are obtained and evaluated in the same manner as for the test samples. The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test patient). The values obtained from the control samples are statistically processed using any suitable method of statistical analysis to establish a suitable baseline level using methods standard in the art for establishing such values.

It will be appreciated by those of skill in the art that a control level of PEG10 or UBQLN2 protein present in a sample need not be established for each assay as the assay is performed but rather, a baseline or control can be established by referring to a form of stored information regarding a previously determined control level for sensitive and resistant patients (responders and non-responders), such as a control level established by any of the above-described methods. Such a form of stored information can include, for example, but is not limited to, a reference chart, listing or electronic file of population or individual data regarding sensitive and resistant/patients, or any other source of data regarding control level that is useful for the patient to be evaluated.

In one embodiment of the present invention, the method includes a step of detecting the expression of a protein, and preferably a PEG10 or UBQLN2 protein. Protein expression can be detected in suitable biological samples. For example, the patient sample, which can be immobilized, can be contacted with an antibody, an antibody fragment, or an aptamer, that selectively binds to the protein to be detected, and determining whether the antibody, fragment thereof or aptamer has bound to the protein. PEG10 protein expression can be measured using a variety of methods standard in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, proteomic mass spectrometry, matrix-assisted laser desorption/ionization time-of-flight (MALDI-TOF) mass spectrometry, microcytometry, microarray, microscopy, fluorescence activated cell sorting (FACS), and flow cytometry. In a preferred embodiment, immunohistochemical (IHC) analysis is used to detect protein expression. IHC methods and preferred assessment criteria for detection of protein expression are described in detail, for example, in Hirsch et al., J. Clin. Oncol. 2003, 21:3798-3807,

In one embodiment of the present invention, the method includes an additional step of detection of a mutation in PEG10 or UBQLN2 genes, and in particular a mutation that result in the accumulation of PEG10 in a cell. In some embodiments, the invention may include the detection of one or more mutations that reduce or eliminate function of the PEG10 or UBQLN2 proteins. Detection of one or more mutations in these genes is predictive that a patient is susceptible to developing ALS, and would likely to benefit from administration of an anti-ALS compound or intervention. Methods for screening for gene mutations are well-known in the art, are described in Lynch et al. and Paez et al., and include, but are not limited to, hybridization, polymerase chain reaction, polyacrylamide gel analysis, chromatography or spectroscopy, and can further include screening for an altered protein product encoded by the gene (e.g., via immunoblot (e.g., Western blot), enzyme-linked immunosorbant assay (ELISA), radioimmunoassay (RIA), immunoprecipitation, immunohistochemistry, immunofluorescence, fluorescence activated cell sorting (FACS) and immunofluorescence microscopy).

The steps of detection of the biomarkers according to the present invention may be combined in many different combinations as described herein, and the steps can be performed in any order, or substantially simultaneously. Statistical analysis to determine differences between controls and patient samples can be performed using any methods known in the art, including, but not limited to, Fisher's exact test of Pearson's chi-square test for qualitative variables, and using Student's t test or analysis of variance for continuous variables. Statistical significance is typically defined as p<0.05.

Another embodiment of the invention includes an assay kit for performing any of the methods of the present invention. The assay kit can include any one or more of the following components: (a) a means for detecting in a sample of ALS cells a level of expression of the PEG10 or UBQLN2 protein. The assay kit preferably also includes one or more controls. The controls could include: (i) information containing a predetermined control level of particular biomarker to be measured with regard to susceptibility to ALS (e.g., a predetermined control level of gene protein expression that has been correlated with susceptibility to ALS in a subject).

In one embodiment, a means for detecting gene or protein expression can generally be any type of reagent that can be used in a method of the present invention. Such a means for detecting include, but are not limited to: a probe or primer(s) that hybridizes under stringent hybridization conditions to a PEG10 or UBQLN2 protein gene. Additional reagents useful for performing an assay using such means for detection can also be included, such as reagents for performing in situ hybridization, reagents for detecting fluorescent markers, reagents for performing polymerase chain reaction, etc.

In another embodiment, a means for detecting PEG10 or UBQLN2 protein expression can generally be any type of reagent that can be used in a method of the present invention. Such a means for detection includes, but is not limited to, antibodies and antigen binding fragments thereof, peptides, binding partners, aptamers, enzymes, and small molecules. Additional reagents useful for performing an assay using such means for detection can also be included, such as reagents for performing immunohistochemistry or another binding assay.

The means for detecting of the assay kit of the present invention can be conjugated to a detectable tag or detectable label. Such a tag can be any suitable tag which allows for detection of the reagents used to detect the gene or protein of interest and includes, but is not limited to, any composition or label detectable by spectroscopic, photochemical, electrical, optical or chemical means. Useful labels in the present invention include: biotin for staining with labeled streptavidin conjugate, magnetic beads (e.g., DYNABEADS™), fluorescent dyes (e.g., fluorescein, Texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

In addition, the means for detecting of the assay kit of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide). The kit can also include suitable reagents for the detection of the reagent and/or for the labeling of positive or negative controls, wash solutions, dilution buffers and the like. The kit can also include a set of written instructions for using the kit and interpreting the results.

The kit can also include a means for detecting a control marker that is characteristic of the cell type being sampled can generally be any type of reagent that can be used in a method of detecting the presence of a known marker (at the nucleic acid or protein level) in a sample, such as by a method for detecting the presence of a biomarker described previously herein. Specifically, the means is characterized in that it identifies a specific marker of the cell type being analyzed that positively identifies the cell type. For example, in a ALS assay, it is desirable to cells for the level of the biomarker expression and/or biological activity. The means for detecting a control marker include, but are not limited to: a probe that hybridizes under stringent hybridization conditions to a nucleic acid molecule encoding a protein marker; PCR primers which amplify such a nucleic acid molecule; an aptamer that specifically binds to a conformationally-distinct site on the target molecule; and/or an antibody, antigen binding fragment thereof, or antigen binding peptide that selectively binds to the control marker in the sample. Nucleic acid and amino acid sequences for many cell markers are known in the art and can be used to produce such reagents for detection. The assay kits and methods of the present invention can be used not only to identify patients that are predicted to be susceptible to ALS or are responsive to a particular anti-ALS compound.

Using the reagents provided in the kits, PEG10 or UBQLN2 activity or expression may be indicated by the observance of one or more (e.g., two, three, four, five, or six) of following features: increased (e.g., by at least 10%, 20%, 30%, 40%, 50%, 60%, 70%, 80%, or 90%) PEG10 or UBQLN2 protein in the biological sample, increased or decreased activity (e.g., by at least 10%, 20%, 30%, 40%, 50%, 60%, 70%, 80%, or 90%) PEG10 or UBQLN2 in the biological sample.

In certain embodiments of the inventive technology, target or market proteins, such as PEG10 or UBQLN2, may encompass the “full protein,” or one or more protein fragments. The methods of the present invention may be used to evaluate fragments of the listed molecules as well as molecules that contain an entire listed molecule, or at least a significant portion thereof (e.g., measured unique epitope), and modified versions of the proteins. Accordingly, such fragments, larger molecules and modified versions are included within the scope of the invention. For example, the target molecules PEG10 or UBQLN2, and their associated signal transduction pathways may include a target protein, protein fragment, epitope, catalytic site, signaling site, localization site and the like.

In certain embodiments of the inventive technology, target or marker genes, such as PEG10 or UBQLN2, may encompass the “full gene,” or one or more gene fragments. The methods of the present invention may be used to evaluate fragments of the listed genes as well as the encoded molecules that contain an entire listed molecule, or at least a significant portion thereof (e.g., measured unique epitope), and modified versions of the genes. Accordingly, such genes or fragments are included within the scope of the invention. For example, the target genes PEG10 or UBQLN2, and their associated genes that may be co-expressed or activated forming one or more signal pathways.

As used herein, a biological marker (“biomarker” or “marker”) is a characteristic that is objectively measured and evaluated as an indicator of normal biologic processes, pathogenic processes, or pharmacological responses to therapeutic interventions, consistent with NIH Biomarker Definitions Working Group (1998). Markers can also include patterns or ensembles of characteristics indicative of particular biological processes. The biomarker measurement can increase or decrease to indicate a particular biological event or process. In addition, if the biomarker measurement typically changes in the absence of a particular biological process, a constant measurement can indicate occurrence of that process.

By “PEG10 levels” or “PEG10 expression” is meant the amount of PEG10 protein, or discrete gag or pol subunits present in a cell (e.g., a biological sample or a control sample).

By “PEG10 protein” is meant a protein that is substantially identical to all or a part of SEQ ID NO. 1, or its discrete gag or pol subunits or any protein having between 80-99% sequence homology with PEG10.

By “UBQLN2 levels” or “UBQLN2 expression” is meant the amount of UBQLN2 protein present in a cell (e.g., a biological sample or a control sample).

By “UBQLN2 protein” is meant a protein that is substantially identical to all or a part of SEQ ID NO. 2, or any protein having between 80-99% sequence homology with UBQLN2.

By “UBQLN2 activity” is meant the activity of UBQLN2, which may be affected by one or mutations that may inhibit or alter its normal biological activity.

By “treating” a disease, disorder, or condition is meant delaying an initial or subsequent occurrence of a disease, disorder, or condition; increasing the disease-free survival time between the disappearance of a condition and its reoccurrence; stabilizing or reducing one or more (e.g., two, three, four, or five) adverse symptom(s) associated with a condition; or inhibiting, slowing, or stabilizing the progression of a condition. The term “treating” also includes reducing (e.g., by at least 5%, 10%, 15%, 20%, 25%, 30%, 35%, 40%, 45%, 50%, 55%, 60%, 65%, 70%, 75%, 80%, 85%, 90%, or 95% the severity or duration of one or more (e.g., one, two, three, four, or five) symptoms of a disease (e.g., ALS) in a patient. Desirably, at least 20%, 40%, 60%, 80%, 90%, or 95% of the treated subjects have a complete remission in which all evidence of the disease disappears. In another desirable embodiment, the length of time a patient survives after being diagnosed with a condition and treated using the methods of the invention is at least 20%, 40%, 60%, 80%, 100%, 200%, or even 500% greater than (i) the average amount of time an untreated patient survives or (ii) the average amount of time a patient treated with another therapy survives.

As used herein, the term “prevent” or “preventing” refers to precluding, averting, obviating, forestalling, stopping, or hindering something from happening, especially by advance action. In an aspect, prevent or preventing refers to the ameliorating of one or more signs and symptoms associated with ALS or FTD. It is understood that where reduce, inhibit or prevent are used herein, unless specifically indicated otherwise, the use of the other two words is also expressly disclosed.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A “therapeutically effective amount” of a compound of a pharmaceutical composition thereof is an amount sufficient to provide a therapeutic benefit in the treatment of a disease or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, and/or enhances the therapeutic efficacy of another therapeutic agent. A “therapeutically effective amount” may also mean “prophylactically effective amount” of a compound of the present invention is an amount sufficient to prevent a disease or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

Administration of a treatment may be effected by any method that enables delivery of the compositions to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the composition and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The term “subject” refers to any animal. In certain embodiments, the subject is a mammal. In certain embodiments, the subject is a human (e.g., a man, a woman, or a child). The human may be of either sex, or may be at any stage of development. In certain embodiments, the subject has been diagnosed with the neurodegenerative condition or disease to be treated. In other embodiments, the subject is at risk of developing the neurodegenerative condition or disease, such as ALS. In certain embodiments, the subject is an experimental animal (e.g., mouse, rat, rabbit, dog, pig, or primate). The experimental animal may be genetically engineered. In certain embodiments, the subject is a domesticated animal (e.g., dog, cat, bird, horse, cow, goat, sheep, or chicken).

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the term “contacting” means bringing together of two elements in an in vitro system or an in vivo system. For example, “contacting” a compound disclosed herein with an individual or patient or cell includes the administration of the compound to an individual or patient, such as a human, as well as, for example, introducing a compound into a sample containing a cellular or purified preparation containing the compounds or pharmaceutical compositions disclosed herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The invention is further described with reference to the following non-limiting Examples.

### EXAMPLES

### Example 1: UBQLN2 Exclusively Regulates the Frameshifted Gag-Pol PEG10

UBQLN2 is one of five human Ubiquilin (UBQLN) genes which facilitate proteasomal degradation of ‘client’ proteins (Hjerpe et al., 2016; Itakura et al., 2016; Lee and Brown, 2012; Suzuki and Kawahara, 2016; Whiteley et al., 2017; Zheng et al., 2020) via an N terminal protein domain which binds to the proteasome 1 (Finley, 2009; Saeki, 2017), and a C-terminal domain which binds to ubiquitin (Zhang et al., 2008). All five UBQLNs have similar protein domain architecture and amino acid sequences, leading to the hypothesis that UBQLNs may have shared client populations. However, UBQLN2 is unique for its enriched expression in neural tissues (Marin, 2014) and for containing a small, proline-rich PXX repeat region that is commonly mutated in UBQLN2-mediated fALS (Deng et al., 2011). To test the specificity of the relationship between UBQLN2 and PEG10, human embryonic stem cells (hESCs) lacking UBQLN1, UBQLN2, or UBQLN4 genes were probed by western blot for endogenous PEG10 protein expression (FIG. 1A). Only UBQLN2−/− hESCs demonstrated an increase in PEG10 protein; furthermore, only the gag-pol form of PEG10 accumulated, while the gag form remained unchanged (FIG. 1B-C).

As UBQLN2 selectively regulated the gag-pol form of PEG10, we hypothesized that a unique region of the pol domain rendered it dependent on UBQLN2 for its degradation. The gag region contains a retroviral capsid domain and CCHC-type zinc finger (FIG. 1D). The pol region of PEG10 is less well understood, but contains an aspartic protease domain (Clark et al., 2007) and a 27 AA C-terminal polyproline repeat (PPR) region containing twelve prolines in tandem, and 18 in total (Clark et al., 2007) (FIG. 1D). To identify the region of PEG10 that defines its reliance on UBQLN2, either PEG10 gag-pol, or a construct lacking the C-terminal PPR, was fused to the fluorescent protein Dendra2 (Klementieva et al., 2016), followed by an IRES-CFP cassette, to generate a transfection-controlled measure of protein abundance (FIG. 1E). PEG10-reporter constructs were then transfected into WT and UBQLN1, 2, and 4 triple knockout (‘TKO’) HEK293 cells (Itakura et al., 2016) to examine the abundance of PEG10 upon UBQLN deficiency (FIG. 1F). While gag-pol protein was more abundant in TKO cells compared to WT cells, PEG10 lacking the PPR failed to accumulate (FIG. 1F-H). These results identify the PEG10 PPR as a necessary region for UBQLN2-dependent restriction.

### Example 2: Human PEG10 Gag-Pol Self-Processes Like a Retrotransposon

The highly specific regulation of gag-pol by UBQLN2 led us to examine the unique properties of this protein in more depth. The pol region of PEG10 contains a retroviral aspartic protease domain with a classic ‘DSG’ active site motif (FIG. 1D) (Clark et al., 2007), which in the ancestral Ty3 retrotransposon results in self-cleavage of capsid (CA) and nucleocapsid (NC) protein fragments with distinct functions (Clemens et al., 2011; Kirchner and Sandmeyer, 1993; Larsen et al., 2008; Sandmeyer and Clemens, 2010). Therefore, we explored the hypothesis that PEG10 gag-pol was capable of self-cleavage. Transfection with an HA-tagged form of PEG10 showed that in addition to the expected gag and gag-pol bands, there were two HA-positive lower molecular 1 weight bands, which we hypothesized were products of self-cleavage (FIG. 2A). When the active site aspartate of the PEG10 protease was mutated to alanine to disrupt proteolytic activity (gag-polASG), we observed a total disappearance of the lower molecular weight HA-tagged bands (FIG. 2A), indicating that the protein products were dependent on PEG10 protease activity. Detailed biochemical analysis and bioinformatic prediction were then performed to identify the precise sites of PEG10 self-cleavage. Together, the results suggested that PEG10 cleaves itself in two locations: AA114-115, and AA260-261. The first cleavage halves the capsid region, and the second generates a zinc-finger containing fragment reminiscent of retrotransposon and retroviral nucleocapsids (FIG. 2B).

Traditionally, retrotransposon and retrovirus gag-pol self-cleavage is necessary to complete the viral lifecycle. For example, proteolytic liberation of retrotransposon nucleocapsid from gag is necessary for proper capsid or virus-like particle (VLP) assembly (Larsen et al., 2008; Sandmeyer and Clemens, 2010). The PEG10 gag protein has been shown to form VLPs (Abed et al., 2019; Segel et al., 2021) that resemble those formed by retrotransposons and the gag-like gene Arc/Arg3.1 (Ashley et al., 2018; Pastuzyn et al., 2018); therefore, we hypothesized that PEG10 self-cleavage may be necessary for proper VLP formation and release. PEG10 was overexpressed in cells and VLPs were harvested from the cultured supernatant by ultracentrifugation. Abundance of VLPs was then probed by western blot. Unlike traditional retrotransposons, self-cleavage was not a prerequisite for PEG10 VLP formation, as gag and gag-polASG were capable of releasing VLPs with similar efficiency (FIG. 2C). However, like its retrotransposon ancestors, PEG10 gag-pol was capable of cleaving PEG10 gag in trans (FIG. 2D), which suggested that a large pool of proteolytic products of gag could be generated from gag-pol activity.

Proteolytic self-processing enables novel functions for domains found in the gag and gag-pol polyproteins. For the Ty3 retrotransposon, the liberation of nucleocapsid from gag regulates the localization of capsid assembly (Larsen et al., 2008). We hypothesized that liberated PEG10 nucleocapsid may have similarly unique localization and function following self-cleavage. To test this, individual PEG10 cleavage products were expressed in cells and their localization was examined by confocal microscopy. 1 All PEG10 proteins (gag, gag-pol, CA, and nucleocapsid) were similarly expressed and localized to the cytoplasm. Intriguingly, however, only nucleocapsid was also observed in the nucleus (FIG. 2E). These data suggest that self-processing of PEG10 may reveal novel functions of its proteolytic products.

### Example 3: PEG10 Nucleocapsid Induces Changes in Gene Expression

The nucleocapsid fragment contains a retroviral CCHC-type zinc finger that has been reported to bind DNA (Steplewski et al., 1998) as well as RNA (Abed et al., 2019; Segel et al., 2021). This, paired with the movement of liberated nucleocapsid to the nucleus, raised the possibility that PEG10 self-cleavage may induce unique transcriptional changes. To test this hypothesis, HEK cells were transfected with either PEG10 gag-pol, gag, or nucleocapsid, and changes in gene expression were analyzed by RNA-Seq. Transfection with PEG10 gag-pol induced the most gene expression changes, followed by nucleocapsid, with gag-transfected cells showing the fewest changes compared to control (Table S1). Cluster profiling identified distinct groups of genes differentially regulated by specific PEG10 constructs. The first two groups consisted of genes that changed upon any type of PEG10 overexpression (FIG. 3A), suggesting generalized responses to virus-like protein expression. One example of a gene upregulated by all forms of PEG10 expression was TXNIP, a regulator of oxidative stress, which is also elevated in multiple neurodegenerative conditions (Tsubaki et al., 2020) (FIG. 3B). The largest cluster profile (Group 3) consisted of genes upregulated upon gag-pol and nucleocapsid transfection, but not gag transfection, highlighting the ability of the small nucleocapsid fragment to induce transcriptional changes in a manner similar to full-length gag-pol protein (FIG. 3A).

Pathway analysis of differentially expressed genes also underscored the similarities in gene regulation between gag-pol and nucleocapsid expression. Gag-pol expression resulted in an overrepresentation of pathways including female-specific sex characteristics, consistent with a role of PEG10 in placental development (Abed et al., 2019; Ono et al., 2006), as well as those involved in axon extension and remodeling. Nucleocapsid expression resulted in an even stronger overrepresentation of neuronal pathways, especially pathways involved in axon guidance and extension (FIG. 3C). One notable example of an axon remodeling gene was DCLK1, which was significantly elevated in nucleocapsid and gag-pol, but unchanged in gag-expressing cells (FIG. 3D-E,). Gag expression resulted in fewer transcript changes and did not alter neuronal gene expression, highlighting the unique effects of gag-pol and nucleocapsid. To better understand the transcriptional effects of PEG10 overexpression, splicing differences were examined across gag-pol, gag, and NC expression conditions. Consistent with changes to transcript abundance, both nucleocapsid and gag-pol expression resulted in splicing alteration of 150-200 genes, whereas gag had fewer effects (Table S1). There were no changes to global patterns of transcript splicing upon nucleocapsid expression, nor were there global changes to mRNA trafficking, indicating that the changes to transcriptional abundance are gene specific. Our data suggested a direct link between PEG10 abundance and changes to neuronal gene expression. To explore whether these transcript changes may be playing a role in human disease, we analyzed transcriptional data from post-mortem sALS patient spinal cord samples and observed similarly elevated levels of TXNIP and DCLK1 transcripts (FIG. 3F), suggesting that ALS involves similar pathways of transcriptional disturbance.

### Example 4: PEG10 Gag-Pol Protein is Elevated in Human ALS Tissues

In ALS, UBQLN2 may be dysfunctional in multiple ways. The first is through genetic mutation, which is observed in UBQLN2-mediated fALS (Deng et al., 2011; Williams et al., 2012) and is thought to cause both a loss of degradative function (Chang and Monteiro, 2015; Le et al., 2016) as well as a toxic gain of function by promoting misfolded UBQLN2 self-assembly (Dao et al., 2019; Sharkey et al., 2018, 2020). To test the ability of mutant UBQLN2 to restrain PEG10 gag-pol levels, TKO cells were complemented with either WT UBQLN2 or two known ALS-causing UBQLN2 missense mutant alleles (Deng et al., 2011) and endogenous PEG10 was quantified by western blot (FIG. 4). TKO cells expressing a WT UBQLN2 construct were able to restrain PEG10 gag-pol to the level seen in WT cells (FIG. 4), demonstrating the ability of UBQLN2 to sufficiently regulate gag-pol protein. Consistent with a loss of function phenotype, mutant UBQLN2P506T-expressing cells failed to restrict PEG10 gag-pol levels (FIG. 4), whereas UBQLN2P497H had no effect on gag-pol. In all cases, gag levels were not dramatically elevated by mutant UBQLN2 expression (FIG. 4C).

UBQLN2 can also be dysfunctional in the absence of overt mutation due to incorporation into ALS-associated protein aggregates (Deng et al., 2011; Williams et al., 2012), where its ability to facilitate proteasomal degradation of PEG10 may be impaired. To broadly examine the abundance of PEG10 in human patients, post-mortem lumbar spinal cord was obtained for global proteomic analysis using a tandem mass tagging (TMT) approach for liquid-chromatography mass spectrometry (LC-MS). Samples were generated from two healthy controls, six sALS cases, and one UBQLN2P497H-mediated fALS case. PEG10 is expressed at very low levels in spinal cord lysate, and in our experience is often below the technical limit of detection by LC-MS. Therefore, the samples were supplemented with a carrier channel designed to drive detection of PEG10 peptides despite the unbiased LC-MS approach (FIG. 5A). Overall, 7,465 unique proteins were quantified across our samples. We observed changes in ALS tissues consistent with previously identified synaptic biomarkers of ALS and neurodegeneration, such as a significant reduction of neurogranin (Kvartsberg et al., 2019; Vijayakumar et al., 2019). Seven peptides were identified from the gag region of PEG10, and three peptides were identified from the pol. While gag was not changed in UBQLN2-mediated or sporadic ALS samples (FIG. 5B), peptides originating from PEG10 pol were significantly enriched in ALS compared to healthy controls (FIG. 5C). This was specific to the protein level, as PEG10 transcript counts were not elevated in ALS. When PEG10 gag and pol were considered as unique proteins (2 peptides per protein minimum,), PEG10 pol was among the most upregulated proteins in all ALS cases compared to healthy controls (FIG. 5D). Its abundance was also associated with a decrease in neurogranin and neurofilament medium, highlighting the potential of PEG10 pol as a novel biomarker for ALS. Taken together, the accumulation of PEG10 gag-pol in ALS tissue, paired with our findings of PEG10 self-cleavage and effects on gene expression, suggest that this pathway may represent a novel pathological contribution to 1 the development of ALS.

### Example 5: Materials and Methods

### Cell Lines

WT HEK293 cells and HEK cells lacking UBQLNs 1, 2, and 4 (‘TKO’) were a gift from Dr. Ramanujan Hegde of the Medical Research Council Laboratory of Molecular Biology. WT and TKO HEK293 cells were cultured in Dulbecco's modified Eagle's medium (Invitrogen) supplemented with 1% penicillin/streptomycin (Invitrogen), 1% L-glutamine (R&D Systems, Inc.), and 10% FBS (Millipore Sigma). HEK293 TKO cells stably transfected with doxycycline-(dox-) inducible constructs expressing Myc-UBQLN2, or Myc-UBQLN2^{P497H} or Myc-UBQLN2^{P506T} were provided by Dr. Hegde via Dr. Miguel Prado and are described in Itakura et al. Dox-inducible cells were cultured in DMEM supplemented with 1% penicillin/streptomycin (Invitrogen), 1% L-glutamine (R&D Systems, Inc.), and 10% tet-system approved FBS (Gibco), as contaminating doxycycline in standard FBS was sufficient to stimulate high levels of UBQLN2 expression. Leaky expression of Myc-UBQLN2 constructs in dox-free medium resulted in endogenous levels of tagged UBQLN2 expression.

Human H9 hESCs lacking either UBQLN1, 2, or 4, were generated at the Harvard Medical School Cell Biology Initiative for Genome Editing and Neurodegeneration according to (Whiteley et al., 2021). hESCs were cultured in either E8 (StemCell Technologies) or TeSR-E8 medium (StemCell Technologies) on 6-well tissue culture plates with Matrigel (Corning, lot #0048006). Medium was changed daily. Cells were passaged by treatment in 0.5 mM EDTA (Sigma Aldrich) in sterile PBS (Invitrogen) and replated in media at an approximate 1:6 dilution. The remaining non-passaged cells were washed in D-PBS three times and pelleted at 300×g for analysis.

### Cell Transfection

WT or TKO HEK293 cells were grown to 70% confluency in 12-well plates and transfected with 1 μg plasmid DNA in Lipofectamine 2000 (Invitrogen) and Opti-Mem medium (Invitrogen), according to manufacturer's instructions. After 48 hours, cells were harvested for western blot, qPCR, RNA-Seq, or immunofluorescence.

### Human Tissue Samples

Human tissue samples were acquired from the Target ALS Multicenter Human Postmortem Tissue Core. Unfixed, full-thickness sections of lumbar spinal cord were obtained from two non-neurological controls, one ALS patient with a pathogenic UBQLN2 mutation, and seven sporadic ALS cases. All cases were from females.

### Cloning

All constructs were designed using Gibson or restriction cloning (see list of constructs) and transformed into chemically competent DH5a E. coli cells (Invitrogen). Transformed E. coli were plated on either 50 μg/mL kanamycin (Teknova) or 100 μg/mL carbenicillin (Gold Biotechnology) LB agar (Teknova) plates overnight at 37° C. Single colonies were picked and grown overnight in 5 mL LB Broth (Alfa Aesar) with kanamycin or carbenicillin at 37° C. with shaking at 220 rpm. The following day, shaking cultures were mini-prepped (Zymo) and sent for Sanger Sequencing. Sequence verified samples were then grown in 50 mL LB Broth overnight with appropriate antibiotic at 37° C. with shaking at 220 rpm. 50 mL cultures were midi-prepped (Zymo) for transfection.

### Flow Cytometry

WT and TKO HEK293 cells were transfected in 96-well plates. Cells were harvested 48 hours after transfection in FACS Buffer (D-PBS, 2% FBS, 0.1% Sodium Azide) and analyzed on a BD FACSCelesta. Triplicate wells were transfected within a plate to serve as technical replicates, and experiments were performed four independent times. FlowJo software was used for data analysis. Cells were first gated in the FSC-A vs. SSC-A using the polygon gating tool. Within the ‘cells’ population, CFP positive cells were gated on 405 nm vs. SSC-A. The Dendra Green/CFP parameter was created by deriving a novel parameter of the 488 reference by the 405 reference, and making a logarithmic scale with a minimum of 0.0001 and a maximum of 10. The geometric mean of the custom Dendra Green/CFP parameter from the CFP positive population was exported and used to generate graphs.

### Western Blotting

Cell pellets were centrifuged, washed in PBS and lysed in urea buffer (8 M urea, 75 mM NaCl, 50 mM HEPES pH 8.5, 1× tab cOmplete Mini EDTA-free protease inhibitor cocktail tablet (Sigma Aldrich)). Lysis was performed by vortex and incubation for 15 minutes at room temperature. Lysate was centrifuged for 10 minutes at 21,300×g and the supernatant was collected as sample.

Protein was quantified by BCA (Pierce) and 1× Laemmli sample buffer supplemented with βME (Sigma Aldrich) was added to samples before SDS-PAGE. Samples were run in NuPage MES Running Buffer (Invitrogen) on a 4 to 12% NuPage Bis-Tris gel (Invitrogen) and wet transferred on nitrocellulose membrane (Amersham Protran) for either 90 minutes at 100 V on ice (BioRad Mini-Blot Transfer) or 60 minutes at 10 V (Invitrogen Mini Tank Blot Module).

Membranes were blocked using 1:1 LICOR blocking buffer and 1×TBS (50 mM Tris-Cl pH 7.4, 150 mM NaCl), for 30 minutes at room temperature. Membranes were incubated in primary antibody overnight at 4° C. and washed in 1×TBST (1×TBS, 0.1% Tween, VWR) in three five-minute intervals. Membranes were then incubated in LICOR secondary antibody for 30 minutes in the dark. After 3× more washes, banding patterns were visualized using LICOR Odyssey CLx and data analysis was performed using LICOR ImageStudio Software. Each protein quantification was normalized to the average intensity across all samples in each replicate western blot to correct for technical variation across experiments.

### Virus-Like Particle Isolation

HEK293 cells were plated in a 6-well plate at a density of 4×10⁵ cells per well. 24 hours after plating, cells were transfected and media was replaced 6 hours later. Cultured media was harvested 48 hours after transfection and pre-cleared by centrifugation at 2000×g for 15 minutes at 4° C. In parallel, cell lysate was collected for western blot as previously described. The VLP fraction was isolated by ultracentrifugation (Beckman Coulter) at 134,000×g for 4 hours with a 30% sucrose cushion. After ultracentrifugation, media and sucrose were aspirated, and the VLP-containing pellet was resuspended in 8M urea lysis buffer. VLP production was analyzed by western blot.

### Structure Prediction

Structure prediction for the PEG10 gag protein (AA1-325) was performed using the Phyre 2.0 webserver (Kelley et al., 2015) using the intensive modeling mode. 243 of 325 amino acids were modeled with >90% confidence, with amino acids 89-314 predicted with confidence >99% against reference structures including Saccharomyces Ty3, Drosophila and Rattus Arc, HIV, and a partial structure of Homo sapiens PEG10 gag. The predicted PEG10 structure was visualized using UCSF Chimera.

### Immunofluorescence

24 hours after transfection, HEK293 cells were re-plated onto Alcian Blue- (Newcomer Supply) treated round coverslips (Electron Microscopy Sciences) in 24-well plates and cultured overnight at 37° C. 24 hours later, coverslips were harvested and fixed in 4% PFA (Thermo Scientific Pierce). Cells were then either submerged in 1% PFA for overnight storage or washed three times in 1×PBS. Cells were permeabilized in 0.25% Triton-X (Sigma Aldrich) in PBS and incubated in blocking buffer (7.5% BSA (Gibco) diluted to 5% in PBS, 0.1% tween) for 30 minutes. Cells were incubated for 1 hour in primary antibody before three 5-minute washes in 1×PBS-T (0.1% Tween in PBS). Cells were then incubated in secondary antibody for one hour in the dark. Cells underwent three more 5-minute 1×PBS-T washes and were then rinsed three times in DEPC water. After sufficient drying, 5-10 mL of Prolong Gold DAPI anti-fade mounting media (Invitrogen) was added to coverslips. Coverslips were then mounted on clear microscope slides and cured overnight in the dark at room temperature before imaging.

### Oligo dT Fluorescence In Situ Hybridization

Transfected WT HEK293 cells were cultured on round coverslips (Electron Microscopy Sciences) and hybridized according to Stellaris protocol for hybridization of adherent cells (Biosearch Technologies). A T30 Poly A probe (Stellaris, Biosearch Technologies) was used to detect polyA mRNA tails as a measure of total mRNA by cellular compartment. 10 images were obtained for each transfection condition and randomly assigned image names for blind quantification. Distinct single cells were quantified using FIJI software XOR function to quantify mean signal intensity of nucleus and cytoplasm. Nucleus to cytoplasmic ratio was calculated for a minimum of 60 cells for each condition.

### Confocal Microscopy

Microscopy was performed on a Nikon AR1 LSM confocal microscope maintained by the BioFrontiers Advanced Light Microscopy Core using a 20× Air objective and NIS Elements Nikon software. Acquisition intensity and pinhole size were fixed across samples to control for signal intensity and variability. For visualization purposes only, image intensity of visualized channels was increased from acquisition parameters according to FIJI software parameters.

### Sample Preparation for RNA Sequencing

HEK293 cells were grown in 12-well plates, transfected for overexpression of genes of interest, and collected for RNA isolation 48 hours later. Cells were pelleted and RNA was extracted using the RNEasy Mini Kit (Qiagen) with on-column DNAse digestion (Qiagen). Isolated RNA was quantified and quality controlled by nanodrop, concentration was normalized, and samples were stored at −80° C.

### RNA-Sequencing Analysis

Poly A Selected Total RNA Library paired-end sequencing was performed at Anschutz Medical Campus on an Illumina NovaSEQ 6000. Sequencing produced between 24-104 million filtered paired end reads across all samples. Quality of reads was determined using FastQC (the average reads/base quality for all samples in the lane was at least 88%>=Q30) and reads were mapped to GRCh38.p13 (Frankish et al., 2019) using STAR version 2.7.3 (Dobin et al., 2013). STAR alignment .bam files were indexed and sorted before count matrix generation using Samtools 1.8 and featureCounts software package (Li et al., 2009).

Count files were converted to readable format in unix and imported into RStudio for DESeq2 analysis using R. Data were quality controlled by estimating size factors and genewise dispersion estimates for variance in gene expression. Shrinking was used to fit dispersion curves and principal component analyses dictated design parameters for differential gene expression analysis. Gene expression patterns were tracked using DESeq2 (Love et al., 2014) using harvest date and transfection construct as major variables, as well as Cluster Profiling (Yu et al., 2012), and GO Term expression (Luo and Brouwer, 2013; Yu et al., 2015) analyses. Significance of gene expression changes was determined with a p-adjusted cutoff of 0.05. Gene groups were determined with DEGReport (Pantano, 2021) using a reduced cluster model in which outliers of cluster distribution were removed.

Pathway analysis was performed using the enrichGO program (Yu et al., 2015) on all GO-term pathways with a log 2foldchange cutoff of 0.5 and a p value of 0.05 of significantly changed genes for each pairwise analysis (pCDNA negative control vs. gag-pol, vs. gag, and vs. NC). The top 5 pathways by p value were visualized.

Splicing analysis was performed using the MAJIQ Quantifier followed by the MAJIQ Builder to determine differentially spliced genes (Vaquero-Garcia et al., 2016), and visualized using the MAJIQ Voila Viewer with a Δψ threshold of 0.1 and significance of 0.05. Splice variant classification analysis was performed using the MAJIQ classifier (Vaquero-Garcia et al., 2021) with permission and assistance from Dr. Yoseph Barash.

### Target ALS Dataset RNA-Seq Analysis

Raw RNA-Seq reads from lumbar spinal cord of the Target ALS: New York Genome Center dataset were obtained; at the time of analysis, this dataset included 40 Classical/Typical ALS cases, 5 non-neurological controls, and multiple samples from other neurodegenerative diseases for a total of 51 samples. Approximately half of the samples were male/female. Reads were aligned to the human genome (hg38) using STAR version 2.5.2b as above (Dobin et al., 2013), and analyzed in RStudio with DESeq2 (Love et al., 2014) including sex and ‘Subject.Group.Subcategory’ (disease type) as major variables. Significance of gene expression changes was determined with a p-adjusted cutoff of 0.05, and normalized counts were used for visualization of target genes.

### Sample Preparation for Mass Spectrometry Analysis

Human spinal cord samples were first sectioned on a cryostat (Leica) to ensure even tissue representation of protein sample. Ten to twenty 15 μm-thickness sections from each patient were homogenized in 8M urea lysis buffer, lysate was spun at 15,000 rpm for 15 minutes at 4° C. to remove insoluble material, and supernatant protein content was quantified by BCA analysis (Pierce). Separately, HEK cells were transfected with Homo sapiens HA-PEG10 gag-pol, lysed 48 hours later, and mixed in a 95:5 ratio of sALS spinal cord lysate to HEK cell lysate. Approximately 100-200 μg of each sample was aliquoted and delivered to the Proteomics and Mass Spectrometry Core Facility in the Department of Biochemistry at the University of Colorado, Boulder, for TMT labeling.

Human lumbar spinal cord tissue samples in 8M urea were reduced and alkylated with the addition of 5% (w/v) sodium dodecyl sulfate (SDS), 10 mM tris(2-carboxyethylphosphine) (TCEP), 40 mM 2-chloroacetamide, 50 mM Tris-HCl, pH 8.5 and incubated shaking at 1000 rpm at room temperature for 60 minutes then cleared via centrifugation at 17,000×g for 10 minutes at 25° C. Lysates were digested using the SP3 method (Hughes et al., 2014). Briefly, 200 μg carboxylate-functionalized speedbeads (Cytiva Life Sciences) were added to approximately 100 μg protein lysate. Addition of acetonitrile to 80% (v/v) induced binding to the beads, then the beads were washed twice with 80% (v/v) ethanol and twice with 100% acetonitrile. Proteins were digested in 50 mM Tris-HCl buffer, pH 8.5, with 1 μg Lys-C/Trypsin (Promega) and incubated at 37° C. overnight. Tryptic peptides were desalted using HLB Oasis 1 cc (10 mg) cartridges (Waters) according to the manufactures instructions and dried in a speedvac vacuum centrifuge.

Approximately 30 μg of tryptic peptide from each human tissue sample was labeled with TMT 10 plex (Thermo Scientific) reagents according to the manufacturer's instructions. The multiplexed sample was cleaned up with a HLB Oasis 1 cc (10 mg) cartridge. Approximately 50 multiplexed peptides were fractionated with high pH reversed phase C18 UPLC using a 0.5 mm×200 mm custom packed UChrom C18 1.8 μm 120 Å (nanolcms) column with mobile phases 10 mM aqueous ammonia, pH10 in water and acetonitrile (ACN). Peptides were gradient eluted at 20 μL/minute from 2 to 40% ACN in 40 minutes concatenating for 12 fractions using a Waters M-class UPLC (Waters). Peptide fractions were then dried in a speedvac vacuum centrifuge and stored at −20° C. until analysis.

### Mass Spectrometry Analysis

High pH peptide fractions were suspended in 3% (v/v) ACN, 0.1% (v/v) trifluoroacetic acid (TFA) and approximately 1 μg tryptic peptides were directly injected onto a reversed-phase C18 1.7 μm, 130 Å, 75 mm×250 mm M-class column (Waters), using an Ultimate 3000 nanoUPLC (Thermos Scientific). Peptides were eluted at 300 nL/minute with a gradient from 4% to 16% ACN over 120 minutes then to 25% ACN in 5 minutes and detected using a Q-Exactive HF-X mass spectrometer (Thermo Scientific). Precursor mass spectra (MS1) were acquired at a resolution of 120,000 from 350 to 1500 m/z with an automatic gain control (AGC) target of 3E6 and a maximum injection time of 50 milliseconds. Precursor peptide ion isolation width for MS2 fragment scans was 0.7 m/z with a 0.2 m/z offset, and the top 15 most intense ions were sequenced. All MS2 spectra were acquired at a resolution of 45,000 with higher energy collision dissociation (HCD) at 32% normalized collision energy. An AGC target of 1E5 and 100 milliseconds maximum injection time was used. Dynamic exclusion was set for 20 seconds with a mass tolerance of ±10 ppm. Raw files were searched against the Uniprot Human database UP000005640 downloaded Nov. 2, 2020 using MaxQuant v.1.6.14.0. Cysteine carbamidomethylation was considered a fixed modification, while methionine oxidation and protein N-terminal acetylation were searched as variable modifications. All peptide and protein identifications were thresholded at a 1% false discovery rate (FDR).

For visualization of data, likely contaminants, reverse peptides, and proteins quantified by only one peptide were removed. P values were calculated by Student's t-test (unpaired, homoscedastic variance) combining both non-neurological control samples and combining all ALS cases (including sporadic and UBQLN2-mediated).

### Statistical Analysis

For western blots, values (normalized to Tubulin and batch-corrected) were compared using the appropriate statistical test by determining normality using a Shapiro-Wilk test and variance using Bartlett's test. For values that were normally distributed and had equal variance, a standard one-way ANOVA was first used to compare differences between the means across all groups. If all groups were not normally distributed a Kruskal-Wallis test was used. Appropriate multiple comparisons tests were utilized to determine which groups significantly varied. For normal distributions, a Bonferroni's multiple comparisons test was used to compare means directly. For non-normally distributed results, a Dunn's multiple comparisons test was used.

For proteomic analysis, values were compared with an unpaired t-test and a threshold of p<0.05 was used for significance. For RNA-Seq data, statistical analysis is described above using DESeq2 and adjusted p values.

For all figures, statistical tests are listed in the figure legend and *p<0.05, **p<0.01, ***p<0.001, and ****p<0.0001.

### REFERENCES

1. Abed, M., Verschueren, E., Budayeva, H., Liu, P., Kirkpatrick, D. S., Reja, R., Kummerfeld, S. K, Webster, J. D., Gierke, S., Reichelt, M., et al. (2019). The Gag protein PEG10 binds to RNA and regulates trophoblast stem cell lineage specification. PLoS ONE 14, e0214110.
2. Akamatsu, S., Wyatt, A. W., Lin, D., Lysakowski, S., Zhang, F., Kim, S., Tse, C., Wang, K., Mo,
3. F., Haegert, A., et al. (2015). The Placental Gene PEG10 Promotes Progression of Neuroendocrine Prostate Cancer. Cell Reports 12, 922-936.
4. Ashley, J., Cordy, B., Lucia, D., Fradkin, L. G., Budnik, V., and Thomson, T. (2018). Retrovirus-like Gag Protein Arc1 Binds RNA and Traffics across Synaptic Boutons. Cell 172, 262-274.e11.
5. Blokhuis, A. M., Groen, E. J. N., Koppers, M., van den Berg, L. H., and Pasterkamp, R. J. (2013). Protein aggregation in amyotrophic lateral sclerosis. Acta Neuropathol 125, 777-794.
6. Brandt, J., Veith, A. M., and Volff, J.-N. (2005). A family of neofunctionalized Ty3/gypsy retrotransposon genes in mammalian genomes. Cytogenet Genome Res 110, 307-317.
7. Brown, R. H., and Al-Chalabi, A. (2017). Amyotrophic Lateral Sclerosis. N Engl J Med 377, 162-172.
8. Chang, L., and Monteiro, M. J. (2015). Defective Proteasome Delivery of Polyubiquitinated Proteins by Ubiquilin-2 Proteins Containing ALS Mutations. PLoS ONE 10, e0130162.
9. Clark, M. B., Jänicke, M., Gottesbühren, U., Kleffmann, T., Legge, M., Poole, E. S., and Tate, W. P. (2007). Mammalian Gene PEG10 Expresses Two Reading Frames by High Efficiency −1 Frameshifting in Embryonic-associated Tissues. J. Biol. Chem. 282, 37359-37369.
10. Clemens, K., Larsen, L., Zhang, M., Kuznetsov, Y., Bilanchone, V., Randall, A., Harned, A., DaSilva, R., Nagashima, K., McPherson, A., et al. (2011). The TY3 Gag3 Spacer Controls Intracellular Condensation and Uncoating. Journal of Virology 85, 3055-3066.
11. Dao, T. P., Martyniak, B., Canning, A. J., Lei, Y., Colicino, E. G., Cosgrove, M. S., Hehnly, H., and Castañeda, C. A. (2019). ALS-Linked Mutations Affect UBQLN2 Oligomerization and Phase Separation in a Position- and Amino Acid-Dependent Manner. Structure 27, 937-951.e5.
12. Deng, H. X., Chen, W., Hong, S. T., Boycott, K. M., Gorrie, G. H., Siddique, N., Yang, Y., Fecto, F., Shi, Y., Zhai, H., et al. (2011). Mutations in UBQLN2 cause dominant X-linked juvenile and adult-onset ALS and ALS/dementia. Nature 477, 211-215.
13. Dobin, A., Davis, C. A., Schlesinger, F., Drenkow, J., Zaleski, C., Jha, S., Batut, P., Chaisson, M, and Gingeras, T. R. (2013). STAR: ultrafast universal RNA-seq aligner. Bioinformatics 29, 15-21.
14. Ferraiuolo, L., Kirby, J., Grierson, A. J., Sendtner, M., and Shaw, P. J. (2011). Molecular pathways of motor neuron injury in amyotrophic lateral sclerosis. Nat Rev Neurol 7, 616-630.
15. Finley, D. (2009). Recognition and Processing of Ubiquitin-Protein Conjugates by the Proteasome. Annu. Rev. Biochem. 78, 477-513.
16. Frankish, A., Diekhans, M., Ferreira, A. M., Johnson, R., Jungreis, I., Loveland, J., Mudge, J. M.,
17. Sisu, C., Wright, J., Armstrong, J., et al. (2019). GENCODE reference annotation for the human and mouse genomes. Nucleic Acids Research 47, D766-D773.
18. Gorrie, G. H., Fecto, F., Radzicki, D., Weiss, C., Shi, Y., Dong, H., Zhai, H., Fu, R., Liu, E., Li, S., et al. (2014). Dendritic spinopathy in transgenic mice expressing ALS/dementia-linked mutant UBQLN2. Proc Natl Acad Sci USA 111, 14524-14529.
19. Hjerpe, R., Bett, J. S., Keuss, M. J., Solovyova, A., McWilliams, T. G., Johnson, C., Sahu, I., Varghese, J., Wood, N., Wightman, M., et al. (2016). UBQLN2 Mediates Autophagy-Independent Protein Aggregate Clearance by the Proteasome. Cell 166, 935-949.
20. Hughes, C. S., Foehr, S., Garfield, D. A., Furlong, E. E., Steinmetz, L. M., and Krijgsveld, J. (2014). Ultrasensitive proteome analysis using paramagnetic bead technology. Mol Syst Biol 10, 757.
21. Itakura, E., Zavodszky, E., Shao, S., Wohlever, M. L., Keenan, R. J., and Hegde, R. S. (2016). Ubiquilins Chaperone and Triage Mitochondrial Membrane Proteins for Degradation. Mol Cell 63, 21-33.
22. Kelley, L. A., Mezulis, S., Yates, C. M., Wass, M. N., and Sternberg, M. J. E. (2015). The Phyre2 web portal for protein modeling, prediction and analysis. Nat Protoc 10, 845-858.
23. Kim, S., Thaper, D., Bidnur, S., Toren, P., Akamatsu, S., Bishop, J. L., Colins, C., Vahid, S., and Zoubeidi, A. (2019). PEG10 is associated with treatment-induced neuroendocrine prostate cancer. Journal of Molecular Endocrinology 63, 39-49.
24. Kirchner, J., and Sandmeyer, S. (1993). Proteolytic processing of Ty3 proteins is required for transposition. J Virol 67, 19-28.
25. Klementieva, N. V., Lukyanov, K. A., Markina, N. M., Lukyanov, S. A., Zagaynova, E. V., and Mishin, A. S. (2016). Green-to-red primed conversion of Dendra2 using blue and red lasers. Chem. Commun. 52, 13144-13146.
26. Kvartsberg, H., Lashley, T., Murray, C. E., Brinkmalm, G., Cullen, N. C., Hoglund, K., Zetterberg, H., Blennow, K., and Portelius, E. (2019). The intact postsynaptic protein neurogranin is reduced in brain tissue from patients with familial and sporadic Alzheimer's disease. Acta Neuropathol 137, 89-102.
27. Larsen, L. S. Z., BeliakovaBethell, N., Bilanchone, V., Zhang, M., Lamsa, A., DaSilva, R., Hatfield, G. W., Nagashima, K., and Sandmeyer, S. (2008). Ty3 Nucleocapsid Controls Localization of Particle Assembly. JVI 82, 2501-2514.
28. Le, N. T. T., Chang, L., Kovlyagina, I., Georgiou, P., Safren, N., Braunstein, K. E., Kvarta, M. D., Van Dyke, A. M., LeGates, T. A., Philips, T., et al. (2016). Motor neuron disease, TDP-43 pathology, and memory deficits in mice expressing ALS-FTD-linked UBQLN2 mutations. Proc Natl Acad Sci USA 113, E7580-E7589.
29. Lee, D. Y., and Brown, E. J. (2012). Ubiquilins in the crosstalk among proteolytic pathways. Biological Chemistry 393, 441-447.
30. Li, H., Handsaker, B., Wysoker, A., Fennell, T., Ruan, J., Homer, N., Marth, G., Abecasis, G., Durbin, R., and 1000 Genome Project Data Processing Subgroup (2009). The Sequence Alignment/Map format and SAMtools. Bioinformatics 25, 2078-2079.
31. Love, M. I., Huber, W., and Anders, S. (2014). Moderated estimation of fold change and dispersion for RNA-seq data with DESeq2. Genome Biol 15, 550.
32. Luo, W., and Brouwer, C. (2013). Pathview: an R/Bioconductor package for pathway-based data integration and visualization. Bioinformatics 29, 1830-1831.
33. Lux, H., Flammann, H., Hafner, M., and Lux, A. (2010). Genetic and Molecular Analyses of PEG10 Reveal New Aspects of Genomic Organization, Transcription and Translation. PLoS ONE 5, e8686.
34. Manktelow, E. (2005). Characterization of the frameshift signal of Edr, a mammalian example of programmed −1 ribosomal frameshifting. Nucleic Acids Research 33, 1553-1563.
35. Marin, I. (2014). The ubiquilin gene family: evolutionary patterns and functional insights. BMC Evol Biol 14, 63.
36. Ono, R., Nakamura, K., Inoue, K., Naruse, M., Usami, T., Wakisaka-Saito, N., Hino, T., Suzuki-Migishima, R., Ogonuki, N., Miki, H., et al. (2006). Deletion of Peg10, an imprinted gene acquired from a retrotransposon, causes early embryonic lethality. Nat Genet 38, 101-106.
37. Pandya, N. J., Wang, C., Costa, V., Lopatta, P., Meier, S., Zampeta, F. I., Punt, A. M., Mientjes, E., Grossen, P., Distler, T., et al. (2021). Secreted retrovirus-like GAG-domain-containing protein PEG10 is regulated by UBE3A and is involved in Angelman syndrome pathophysiology. Cell Reports Medicine 2, 100360.
38. Pantano, L. (2021). DEGreport: Report of DEG analysis. R package version 1.30.0, http://lpatano.github.io/DEGreport/. (Bioconductor). Pastuzyn, E. D., Day, C. E., Kearns, R. B., KyrkeSmith, M., Taibi, A. V., McCormick, J., Yoder, N., Belnap, D. M., Erlendsson, S., Morado, D. R., et al. (2018). The Neuronal Gene Arc Encodes a Repurposed Retrotransposon Gag Protein that Mediates Intercellular RNA Transfer. Cell 172, 275-288.e18.
39. Peters, O. M., Ghasemi, M., and Brown, R. H. (2015). Emerging mechanisms of molecular pathology in ALS. J. Clin. Invest. 125, 1767-1779.
40. Riedl, J., Crevenna, A. H., Kessenbrock, K., Yu, J. H., Neukirchen, D., Bista, M., Bradke, F., Jenne, D., Holak, T. A., Werb, Z., et al. (2008). Lifeact: a versatile marker to visualize F-actin. Nat Methods 5, 605-607.
41. Saeki, Y. (2017). Ubiquitin recognition by the proteasome. J Biochem mvw091.
42. Sandmeyer, S. B., and Clemens, K. A. (2010). Function of a retrotransposon nucleocapsid protein. RNA Biology 7, 642-654.
43. Segel, M., Lash, B., Song, J., Ladha, A., Liu, C. C., Jin, X., Mekhedov, S. L., Macrae, R. K., Koonin, E. V., and Zhang, F. (2021). Mammalian retrovirus-like protein PEG10 packages its own mRNA and can be pseudotyped for mRNA delivery. Science 373, 882-889.
44. Sharkey, L. M., Safren, N., Pithadia, A. S., Gerson, J. E., Dulchaysky, M., Fischer, S., Patel, R., Lantis, G., Ashraf, N., Kim, J. H., et al. (2018). Mutant UBQLN2 promotes toxicity by modulating intrinsic self-assembly. Proc Natl Acad Sci USA 115, E10495-E10504.
45. Sharkey, L. M., Sandoval-Pistorius, S. S., Moore, S. J., Gerson, J. E., Komlo, R., Fischer, S., Negron-Rios, K. Y., Crowley, E. V., Padron, F., Patel, R., et al. (2020). Modeling UBQLN2-mediated neurodegenerative disease in mice: Shared and divergent properties of wild type and mutant UBQLN2 in phase separation, subcellular localization, altered proteostasis pathways, and selective cytotoxicity. Neurobiology of Disease 143, 105016.
46. Steplewski, A., Krynska, B., Tretiakova, A., Haas, S., Khalili, K., and Amini, S. (1998). MyEF-3, a Developmentally Controlled Brain-Derived Nuclear Protein Which Specifically Interacts with Myelin Basic Protein Proximal Regulatory Sequences. Biochemical and Biophysical Research Communications 243, 295-301.
47. Suzuki, R., and Kawahara, H. (2016). UBQLN4 recognizes mislocalized transmembrane domain proteins and targets these to proteasomal degradation. EMBO Rep 17, 842-857.
48. Tsubaki, H., Tooyama, I., and Walker, D. G. (2020). Thioredoxin-Interacting Protein (TXNIP) with Focus on Brain and Neurodegenerative Diseases. IDMS 21, 9357.
49. Vaquero-Garcia, J., Barrera, A., Gazzara, M. R., Gonzalez Vallinas, J., Lahens, N. F., Hogenesch,
50. J. B., Lynch, K. W., and Barash, Y. (2016). A new view of transcriptome complexity and regulation through the lens of local splicing variations. ELife 5, e11752.
51. VaqueroGarcia, J., Aicher, J. K., Jewell, P., Gazzara, M. R., Radens, C. M., Jha, A., Green, C. J.,
52. Norton, S. S., Lahens, N. F., Grant, G. R., et al. (2021). RNA splicing analysis using heterogeneous and large RNA-seq datasets (Bioinformatics).
53. Vijayakumar, U. G., Milla, V., Cynthia Stafford, M. Y., Bjourson, A. J., Duddy, W., and Duguez, S. M.-R. (2019). A Systematic Review of Suggested Molecular Strata, Biomarkers and Their Tissue Sources in ALS. Front. Neurol. 10, 400.
54. Volff, J.-N. (2006). Turning junk into gold: domestication of transposable elements and the creation of new genes in eukaryotes. Bioessays 28, 913-922.
55. Whiteley, A. M., Prado, M. A., Peng, I., Abbas, A. R., Haley, B., Paulo, J. A., Reichelt, M., Katakam, A., Sagolla, M., Modrusan, Z., et al. (2017). Ubiquilin1 promotes antigen-receptor mediated proliferation by eliminating mislocalized mitochondrial proteins. Elife 6.
56. Whiteley, A. M., Prado, M. A., dePoot, S. A. H., Paulo, J. A., Ashton, M., Dominguez, S., Weber, M., Ngu, H., Szpyt, J., Jedrychowski, M. P., et al. (2021). Global proteomics of Ubqln2-based murine models of ALS. J. Biol. Chem. 296, 100153.
57. Williams, K. L., Warraich, S. T., Yang, S., Solski, J. A., Fernando, R., Rouleau, G. A., Nicholson, G. A, and Blair, I. P. (2012). UBQLN2/ubiquilin 2 mutation and pathology in familial amyotrophic lateral sclerosis. Neurobiology of Aging 33, 2527.e3-2527.e10.
58. Yu, G., Wang, L.-G., Han, Y., and He, Q.-Y. (2012). clusterProfiler: an R Package for Comparing Biological Themes Among Gene Clusters. OMICS: A Journal of Integrative Biology 16, 284-287.
59. Yu, G., Wang, L.-G., Yan, G.-R., and He, Q.-Y. (2015). DOSE: an R/Bioconductor package for disease ontology semantic and enrichment analysis. Bioinformatics 31, 608-609.
60. Zhang, D., Raasi, S., and Fushman, D. (2008). Affinity Makes the Difference: Nonselective Interaction of the UBA Domain of Ubiquilin-1 with Monomeric Ubiquitin and Polyubiquitin Chains. Journal of Molecular Biology 377, 162-180.
61. Zheng, T., Yang, Y., and Castañeda, C. A. (2020). Structure, dynamics and functions of UBQLNs: at the crossroads of protein quality control machinery. Biochemical Journal

SEQUENCE LISTING
Amino Acid
PEG10
Homo sapiens
SEQ ID NO. 1
MTERRRDELSEEINNLREKVMKQSEENNNLQSQVQKLTEENTTLREQVEP
TPEDEDDDIELRGAAAAAAPPPPIEEECPEDLPEKFDGNPDMLAPFMAQC
QIFMEKSTRDFSVDRVRVCFVTSMMTGRAARWASAKLERSHYLMHNYPAF
MMEMKHVFEDPQRREVAKRKIRRLRQGMGSVIDYSNAFQMIAQDLDWNEP
ALIDQYHEGLSDHIQEELSHLEVAKSLSALIGQCIHIERRLARAAAARKP
RSPPRALVLPHIASHHQVDPTEPVGGARMRLTQEEKERRRKLNLCLYCGT
GGHYADNCPAKASKSSPAGKLPGPAVEGPSATGPEIIRSPQDDASSPHLQ
VMLQIHLPGRHTLFVRAMIDSGASGNFIDHEYVAQNGIPLRIKDWPILVE
AIDGRPIASGPVVHETHDLIVDLGDHREVLSFDVTQSPFFPVVLGVRWLS
THDPNITWSTRSIVFDSEYCRYHCRMYSPIPPSLPPPAPQPPLYYPVDGY
RVYQPVRYYYVQNVYTPVDEHVYPDHRLVDPHIEMIPGAHSIPSGHVYSL
SEPEMAALRDFVARNVKDGLITPTIAPNGAQVLQVKRGWKLQVSYDCRAP
NNFTIQNQYPRLSIPNLEDQAHLATYTEFVPQIPGYQTYPTYAAYPTYPV
GFAWYPVGRDGQGRSLYVPVMITWNPHWYRQPPVPQYPPPQPPPPPPPPP
PPPSYSTL
Amino Acid
UBQLN2
Homo sapiens
SEQ ID NO. 2
MAENGESSGPPRPSRGPAAAQGSAAAPAEPKIIKVTVKTPKEKEEFAVPE
NSSVQQFKEAISKRFKSQTDQLVLIFAGKILKDQDTLIQHGIHDGLTVHL
VIKSQNRPQGQSTQPSNAAGTNTTSASTPRSNSTPISTNSNPFGLGSLGG
LAGLSSLGLSSTNFSELQSQMQQQLMASPEMMIQIMENPFVQSMLSNPDL
MRQLIMANPQMQQLIQRNPEISHLLNNPDIMRQTLEIARNPAMMQEMMRN
QDLALSNLESIPGGYNALRRMYTDIQEPMLNAAQEQFGGNPFASVGSSSS
SGEGTQPSRTENRDPLPNPWAPPPATQSSATTSTTTSTGSGSGNSSSNAT
GNTVAAANYVASIFSTPGMQSLLQQITENPQLIQNMLSAPYMRSMMQSLS
QNPDLAAQMMLNSPLFTANPQLQEQMRPQLPAFLQQMQNPDTLSAMSNPR
AMQALMQIQQGLQTLATEAPGLIPSFTPGVGVGVLGTAIGPVGPVTPIGP
IGPIVPFTPIGPIGPIGPTGPAAPPGSTGSGGPTGPTVSSAAPSETTSPT
SESGPNQQFIQQMVQALAGANAPQLPNPEVRFQQQLEQLNAMGFLNREAN
LQALIATGGDINAAIERLLGSQPS

What is claimed is:

1. An assay kit for determining susceptibility to (amyotrophic lateral sclerosis (ALS) in a subject, the assay kit comprising:
i) at least one means for detecting in a biological sample of the subject a level of PEG10; and,
ii) a control level selected from the group consisting of:
a) a control level for PEG10 indicating susceptibility to the development of ALS;
b) a control level for PEG10 indicating non-susceptibility to the development of ALS;
c) information containing a predetermined control level of the PEG10 that has been correlated with susceptibility to the development of ALS; and
d) information containing a predetermined control level of the PEG10 that has been correlated with non-susceptibility to the development of ALS.

2. The assay kit of claim 1, further comprising at least one means for detecting at least one mutation in the PEG10 and/or UBQLN2 genes.

3. The assay kit of claim 1, further comprising at least one means for detecting in a biological sample of a subject a level of a UBQLN2.

4. The assay kit of claim 1, further comprising at least one means for detecting in a biological sample of a subject the activity level of a UBQLN2.

5. The assay kit of claim 1, further comprising a control level selected from the group consisting of:
a) a control level for UBQLN2 indicating susceptibility to the development of ALS;
b) a control level for UBQLN2 indicating non-susceptibility to the development of ALS;
c) information containing a predetermined control level of the UBQLN2 that has been correlated with susceptibility to the development of ALS; and
d) information containing a predetermined control level of the UBQLN2 that has been correlated with non-susceptibility to the development of ALS.

6. The assay kit of claim 1, further comprising at least one means for detecting in a biological sample of a subject a level of a fragment of PEG10.

7. The assay kit of claim 1, wherein the fragment of PEG10 comprises a gag or pol fragment of PEG10.

8. The assay kit of claim 1, wherein the means for detecting the PEG10 is selected from the group consisting of: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, proteomic mass spectrometry, microcytometry, microarray, microscopy, fluorescence activated cell sorting (FACS), and flow cytometry.

9. The assay kit of claim 1, wherein the biological sample comprises a neuronal cell.

10. The assay kit of claim 1, wherein the biological sample comprises a bodily fluid.

11. The assay kit of claim 10, wherein the bodily fluid comprises cerebrospinal fluid (CNS) from a subject.

12. A method for predicting the clinical response of a subject to an anti-ALS medication comprising obtaining a biological sample from a subject and detecting in the protein levels of PEG10 in said sample and further correlating the level of the biomarker with an increased likelihood for the patient to have a beneficial clinical response to an early ALS intervention.

13. The method of claim 12, and further comprising administering the ALS intervention to said subject.

14. The method of claim 13, wherein the ALS intervention comprise an anti-ALS medication

15. The method of claim 14, wherein the anti-ALS medication is riluzole and/or edaravone.

16. An method for of treating amyotrophic lateral sclerosis (ALS) in a subject, the method comprising:
i) detecting in a biological sample of the subject a level of PEG10 protein; and,
ii) correlating elevated levels PEG10 protein in the sample compared to a control level of PEG10 protein that has been correlated with susceptibility to ALS, with an increased likelihood that the patient will respond to an ALS intervention regimen said anti-ALS medication;
iii) administering said anti-ALS medication to treat ALS in said subject based on the above correlation showing said subject will benefit from the ALS intervention comprising an anti-ALS medication; and
iv) wherein the anti-cancer medication is selected from the group consisting of: riluzole and/or edaravone:

17. The method of claim 16, wherein the level of PEG10 protein comprises the level of gag or pol fragments of PEG10.

18. The method of claim 16, wherein the step of detecting PEG10 is selected from the group consisting of: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, proteomic mass spectrometry, microcytometry, microarray, microscopy, fluorescence activated cell sorting (FACS), and flow cytometry.

19. The method of claim 16, wherein the biological sample comprises a neuronal cell or bodily fluid.

20. The method of claim 19, wherein the bodily fluid comprises cerebrospinal fluid (CNS) from a subject.
